(19) United States
(12) Patent Application Publication
(10) Pub. No.: US 2022/0175326 A1
(43) Pub. Date: Jun. 9, 2022

(54) Non-Invasive Experimental Integrated Reality System

(71) Applicant: Dennis J. Solomon, YARMOUTH PORT, MA (US)

(72) Inventor: Dennis J. Solomon, YARMOUTH PORT, MA (US)

(21) Appl. No.: 17/454,325

(22) Filed: Nov. 10, 2021

Related U.S. Application Data
(63) Continuation-in-part of application No. 16/819,091, filed on Mar. 14, 2020, now Pat. No. 11,199,714.
Application No. 16/819,091 is a continuation-in-part of application No. 16/190,044, filed on Nov. 13, 2018.

(51) Int. Cl. A61B 5/00; G02C 5/00; G02B 27/01; G04B 37/12

(52) U.S. Cl. CPC A61B 5/7445; G02C 5/006; G02B 27/0176; H04M 1/0202; G04B 37/12; A61B 5/6803; A61B 5/681; G02B 27/0172

(57) ABSTRACT

A transformative, collapsible, portable and versatile personal display system is disclosed having a compact, lightweight collapsible structure enabling applications from eyeglass form virtual, augmented, mixed or experimental reality to a comfortable, portable wristwatch form. The system may be employed at public events and manifestations and permit the unique simultaneous transmission to a multiplicity of locations.

### CROSS-REFERENCE TO RELATED APPLICATIONS

This continuation-in-part and divisional application claims the benefit of the earlier filing date and/or incorporates by reference in their entirety my related and earlier-filed applications and disclosures including Ser. No 13/294,011 (pending); Ser. No. 14/189,232 (pending); Ser. No. 16/196,044 (pending) and Ser. No. 16/819,091 (pending); provisional App. No. 63,230,081 filed Aug. 8, 2021 and provisional App. No. 63,222,599 filed Jul. 16, 2021.

The aforementioned benefit of filings are recorded in the submitted ADS.

### TECHNICAL FIELD

The present invention related generally to experimental reality integrated systems which may include elements for creation, editing, monitoring and display of virtual, augmented and mixed reality manifestations for any application, purpose or industry; telecommunications; and optical and personal display systems applied to virtual, augmented and mixed reality systems for all applications, entertainment manifestations and head-mounted displays.

### BACKGROUND ART

Miniature displays are also well known and may involve a miniaturized version of planar or stereoscopic 3D technologies which display a distinct image to each eye. With increase miniaturization and incorporation into eyeglasses design, head-mounted displays (HMDs) have enjoyed an increasing popularity for applications ranging from fighter pilot helmet displays and endoscopic surgery to virtual reality games and augmented reality glasses. The 3D HMD display technology has numerous extensions including Near-to-Eye (NTD)—periscopes and tank sights; Heads-Up (HUD)—windshield and augmented reality—and immersive displays (IMD)—including CAVE, dome and theater size environments. The principal employed varies little from that of the 1930 Polaroid™ glasses, or the barrier stereoscopic displays of the 1890s, despite extensive invention related to the active technology to produce each display has occurred over the past twenty years. As applied to small displays, these techniques evolved to include miniature liquid crystal, field emission, OLED, quantum dot and other two-dimensional matrix displays; variations of virtual screen and retinal scanning methodologies. These inventions have provided practical solutions to the problem of providing lightweight, high resolution displays but are limited to providing a stereoscopic view by means of image disparity.

It is also well known in the field that wavefront-based technologies, such as digital phase and diffractive holography, may at high-resolutions, convey a limited amount of accommodation data. However, their limitations including coherent effects, impart significant specular and other aberrations degrading performance and inducing observer fatigue.

Augmented reality had in origins at MIT Lincoln Laboratory in the 1960s and involved in a translucent HMD with head-orientation tracking in a wall projection immersive environment. The ‘virtual image’ in the HMD did not have accommodation, and the immersive environment did not include spatially-tracked, portable audience elements with multiplicative effects.

Despite the improvements during the past decades, the significant problem of providing a low cost, highly accurate visual display with full accommodation remains.

One of the principal limitations has been the inability of sequentially resonant or programmed variable focal length optics combined with scanning configurations to properly display solid three dimensional pixels, orthogonal to the scanning plane. Another limitation is the inability of the observer's eye to properly and comfortably focus on rapidly flashing elements. Numerous inventions have been proposed which have generally been too complicated to be reliable, too expensive to manufacture, without sufficient resolution, accuracy, stability to gain wide acceptance.

A further problem solved by the innovation of present invention is the method and apparatus to comfortably and useful carry and use an audio-visual display on one's person.

A further problem solved by the innovation of present invention is the method and apparatus to ergonomically, comfortably and useful carry and use an audio-visual display on one's person.

A further problem solved by the innovation of present invention is the method and apparatus to provide lightweight, optical components with high resolution and negligible chromatic aberrations.

A further problem solved by the innovation of present invention is the method and apparatus to provide lightweight, optical components with high resolution and negligible chromatic aberrations which may be transformed into a compact package;

A further problem solved by the innovation of present invention is to provide the method and apparatus which is lightweight, ergonomic, with high resolution and negligible chromatic aberrations, and which may be transformed into a compact package and integrated into an event manifestation.

The present invention solves these and additional problems, particularly related to the portable multiphasic design, augmented reality, environmental dynamics and the accurate display of 3D pixels.

### BRIEF SUMMARY OF THE INVENTION

The present invention discloses an improved method and device for the display of a visual image in two or three dimensions including stereoscopic and/or visual accommodation, light field, beam holographic or diffractive. Another object of the present invention is an improved method and device for an immersive, augmented reality environment.

Another object of the present invention is an improved method and device for monitoring the physiological, psychological, fixation, processing, awareness and response of an individual.

Another object of the present invention is an improved method and device for constructing an accurate, augmented reality, visual display with automatic bi-ocular alignment,

Another object of the present invention is an improved method and device for constructing an accurate, augmented reality, visual display without an intermediate image plane,

Another object of the present invention is an improved method and device for manufacturing a visual display independent of coherence and wavefront curvature constraints,

Another object of the present invention is an improved method and device for thin, wave-guided display.

Another object of the present invention is an improved method of presenting visual information,

Another object of the present invention is an improved method and device for an immersive, augmented-virtual reality, audience performance environment.

Another object of the present invention is an improved method and device to present visual information in compact form unaffected by an external environment.

Another object of the present invention is an improved method and device to compactly wear upon one's person and transform into an immersive, augmented environment.

Another object of the present invention is an improved method and device to compactly wear upon one's person and transform into an immersive, augmented or virtual environment including a coordinated event manifestation and audience effects.

Another object of present invention relates generally to robotic, moving-light devices including those which illuminate and project data and images in visible and invisible wavelengths particularly to those used for theatre, stage, events, security and defense.

One object of the present invention is an improved luminaire, compact in size, lightweight, ad with a low moment of inertia.

Another object is 4π, continuous scan of the venue,

Another object is high efficiency, low cost, low maintenance design without electrical slip rings, split transformers or other devices to transfer base electrical power to a rotating optical element.

Another object is low moment of inertia of the rotating optical projection element,

Another object is a lightweight and compact design.

### BRIEF DESCRIPTION OF THE DRAWINGS

The above and still further objects, features and advantages of the present invention will become apparent upon consideration of the following detailed disclosure of specific embodiments of the invention, especially when taken in conjunction with the accompanying drawings, wherein:

FIG. 1 shows a perspective view of a Wrist Base Station embodiment of the present invention.

FIG. 1A shows a perspective view of a Base Station Element of the present invention.

FIG. 1B shows a perspective view of a Transformative Display Element of the present invention.

FIG. 2 presents a side view of a nanotechnology-embedded embodiment of the present invention.

FIG. 2A presents a side view of an direct center axis, illumination adjustment to a planar nanotechnology-embedded embodiment of the present invention.

FIG. 2B presents a side view of an off-center axis, illumination beam shift adjustment to a planar nanotechnology-embedded embodiment of the present invention.

FIG. 2C presents a side view of an off-center axis, illumination beam shift adjustment to a curved nanotechnology-embedded embodiment of the present invention.

FIG. 3 shows a front view of flexible, transformative display embodiment in the four stage of transformation of the present invention.

FIG. 3A shows a transformative eyeglass to watch display embodiment with adjustable arms of the present invention.

FIG. 3B shows a transformative eyeglass to watch display embodiment with elastic bridges and arms of the present invention.

FIG. 4 shows a collapsible, fold-away virtual reality lens embodiment of the present invention. which may be employed in manifestations of any sort.

FIG. 4A shows a front view of collapsible, fold-away virtual reality lens elements of the present invention.

FIG. 5 shows a top view of a compact, collapsible, head Strap embodiment of the present invention.

FIG. 5A shows a side view of a compact, collapsible, head Strap embodiment of the present invention.

FIG. 6 shows a side view of a game or trade show embodiment of the present invention, wherein the scene visualization means includes a means to redirect the field of view from the orthogonal to a proximal floor.

FIG. 6A shows a side view of a general, game or trade show ergo embodiment of the present invention, wherein the scene visualization means includes a means to redirect the field of view from the orthogonal to an off-axis surface.

FIG. 6B shows a side view of a general, game or trade show classical display embodiment of the present invention, wherein the scene visualization means includes a means to redirect the field of view from the orthogonal to an off-axis surface.

FIG. 6C shows a perspective view of a general, game or trade show classical display embodiment of scene or optical axis redirection elements.

FIG. 7 shows side and front views of versatile Game-Trade Show Name Tag embodiment of the present invention.

FIG. 7A shows the first view of a sequence of three side views of a preferred construction of the versatile Game-Trade Show Name Tag embodiment of the present invention transforming from compact to object view.

FIG. 7B shows a preferred construction of the versatile Game-Trade Show Name Tag embodiment of the present invention in its expanded configuration.

FIG. 7C shows a preferred construction of the versatile Game-Trade Show Name Tag embodiment of the present invention having a rotatable phone component.

FIG. 7D shows a tabletop preferred construction of the versatile Game-Trade Show Name Tag embodiment of the present invention.

FIG. 8 shows a side view of the diffusive overlay to an image camera of the present invention for receiving an optical data signal.

FIG. 9 shows a preferred method of and construction for varying the perceived focal distance of a display element of the present invention.

FIG. 9A shows a preferred method of and construction for enabling a divergent array of light beams to simulate a proximal display element.

FIG. 9B shows a preferred method of and construction for enabling a parallel beam of light to simulate a distal display element.

FIG. 10 presents an interactive embodiment of the present invention which may be employed in manifestations of any sort having the flip-up eye visor with mixed-reality cameras embodiment of the present invention.

FIG. 11 shows a perspective view of a game or trade show embodiment of the present invention, wherein the display visualization means includes an external display area.

FIG. 12 shows a side view of a game or trade show embodiment of the present invention, wherein the display visualization means includes an external display area.

FIG. 13 shows a side view of a compact, foldable embodiment of the present invention exposing the smart phone display.

FIG. 14 a side view of preferred fully Folded Opera embodiment.

FIG. 15A-C shows preferred conforming headstrap embodiments.

FIG. 16A-F show preferred foldable headset construction embodiments.

FIG. 17A-B shows perspective views of foldable headset temple arms embodiments.

FIG. 18 shows a perspective view of a divided display embodiment of the foldable headset.

FIG. 19 shows a perspective view of the vertical Opera sliding enclosure.

FIG. 20 shows a perspective view of a horizontal Opera sliding enclosure.

FIG. 21A-E shows a perspective view of the optical axis redirection assembly.

FIG. 22 shows a perspective view of the optical axis redirection adjustable mirror.

FIG. 23 shows an upper perspective view of the adjustable redirection assembly.

FIG. 24 shows an upper perspective view of the adjustable redirection assembly.

FIG. 25 shows a side view of the adjustable redirection assembly.

FIG. 26 shows a lower perspective view of the adjustable redirection assembly.

FIG. 27 shows an upper perspective view of the slider assembly.

FIG. 28 shows a side view of the rotational race assembly.

FIG. 29 shows a perspective view of the rotational axis assembly.

FIG. 30 shows a shaded perspective view of the rotational axis assembly.

FIG. 31 shows a side schematic view of the ergo redirection configuration.

FIG. 32 shows a side schematic view of the inclined direct redirection configuration.

FIG. 33 shows a side schematic view of the direct redirection configuration.

FIG. 34 shows a side schematic view of the inclined cushioned configuration.

FIG. 35 shows schematic views of the redirection configurations.

FIG. 36 shows a perspective view of the telescoping redirection assembly.

FIG. 37 shows a lower perspective view of the telescoping redirection assembly.

FIGS. 38-40 shows linear accommodation scan projector constructions.

FIG. 41 shows a multi-dimensional arrangement of photonic emitter scan projector.

FIGS. 42-44 shows transcranial monitoring and inducing devices applied.

FIG. 45 shows a general schematic of the audience effects embodiment.

FIGS. 46A-48B show representative embodiments of the audience receiver unit wherein FIGS. 46A and 46B show a “baseball cap” embodiment, FIGS. 47 and 40B an eyeglass-form embodiment, and FIG. 48B split-screen embodiment.

FIGS. 49-51 show the application of collapsible constructions to Wristwatch embodiments.

### DETAILED DESCRIPTION OF THE PRESENT INVENTION

In the following descriptions, the integrated headset device and system 10 may refer to a multiplicity of discrete elements (displays, cameras, touchscreens, computers, motion sensors, rf and optical communications, microphones, speakers, physiological sensors and other elements integrated into a functional structure. Unless specifically described, the descriptions, functions and references may also refer to a well-known “Smart Phone”, manufactured by or marketed as an iPhone®, Samsung®, or others. The interactive embodiments of the present invention may be employed in manifestations of any sort to enable any effects or communication including but not limited to visual, audio streaming and interactivity.

(Base Station)

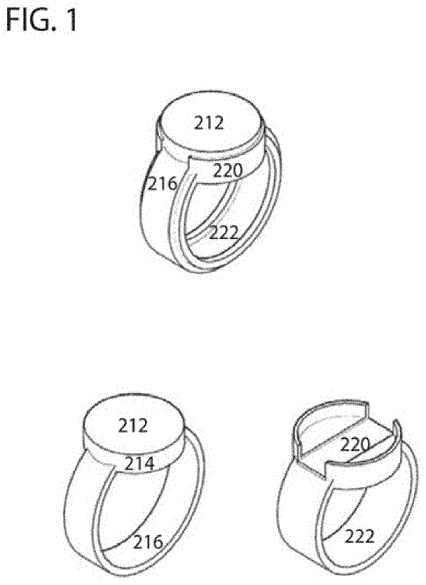

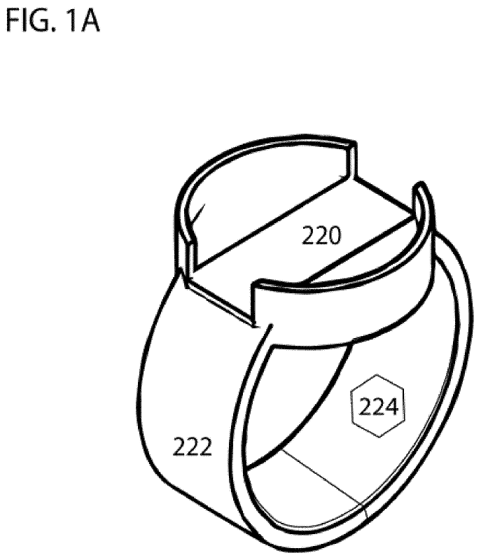

FIG. 1 shows a perspective view of a wrist base station embodiment of the present invention wherein the transformative display elements 212, 214 in the form of a wristwatch-headset 216 may be removably affixed to base station element 220 which may be worn as a bracelet or watch 222. In operation, the base station, in addition to providing a secure location for storage of the transformative display 212 may house an additional charging battery; telecommunications, WiFi and other communications electronics; environmental and user physiology sensors; additional computational elements; and other elements. The base station 220 may communicate with the transformative display elements 212 by short range, low power communications including but not limited to Bluetooth, optical and acoustic means.

FIG. 1A shows a perspective view of a base station element 220 having an physiology sensor 224 on the wristband 222.

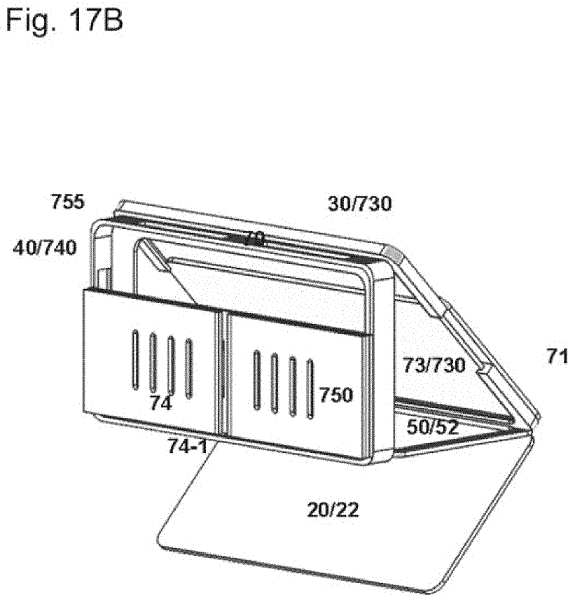

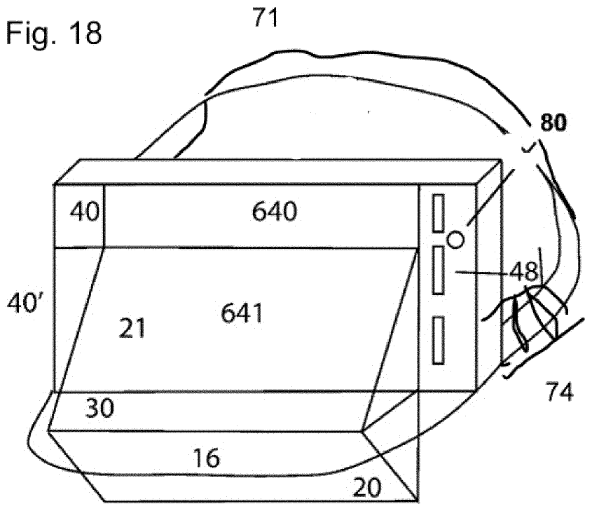

FIG. 18 shows a perspective view of a transformative display element 212, 214 wherein the wristband or base station attaching arms 216 transform in the arms of the headset (eyeglasses).

(Nanotech Embedded Display)

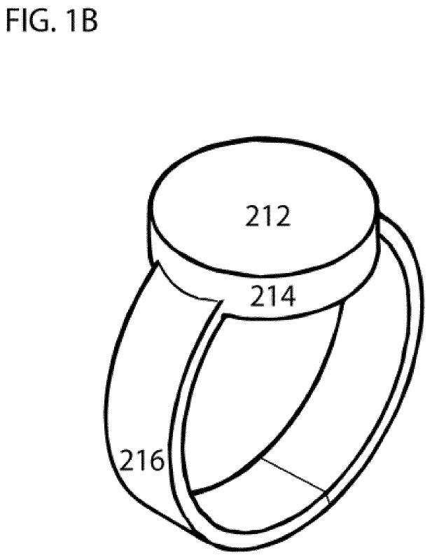

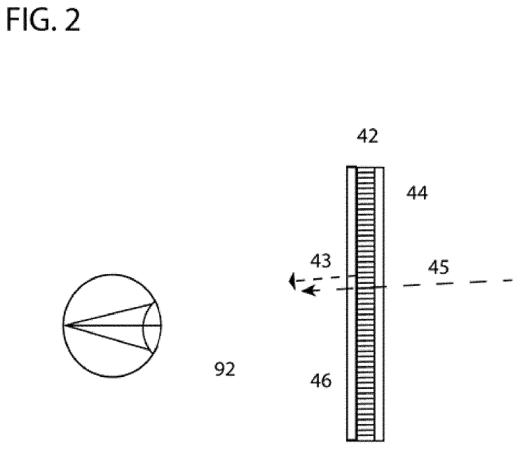

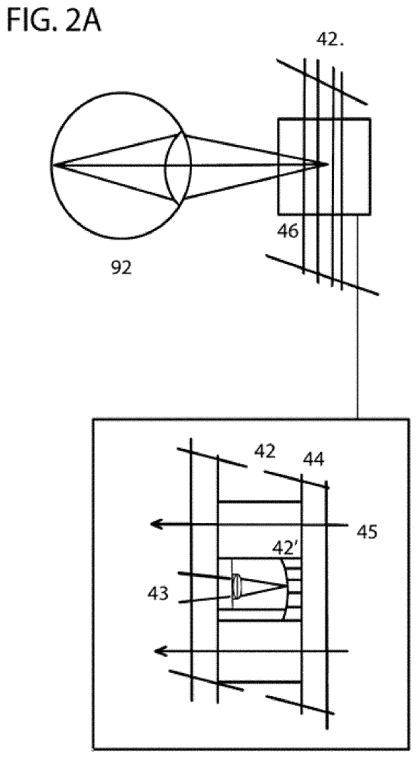

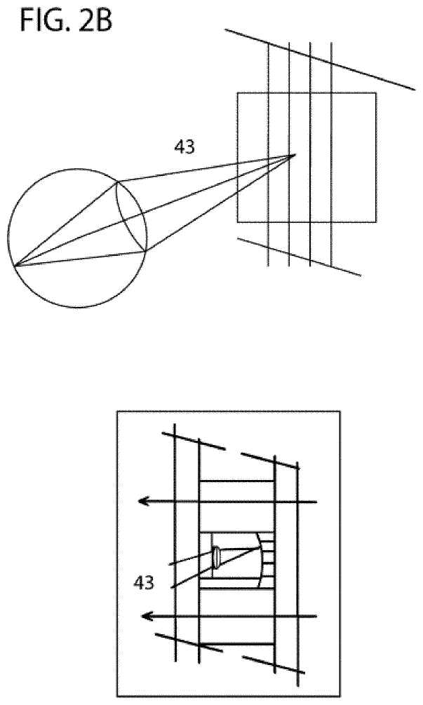

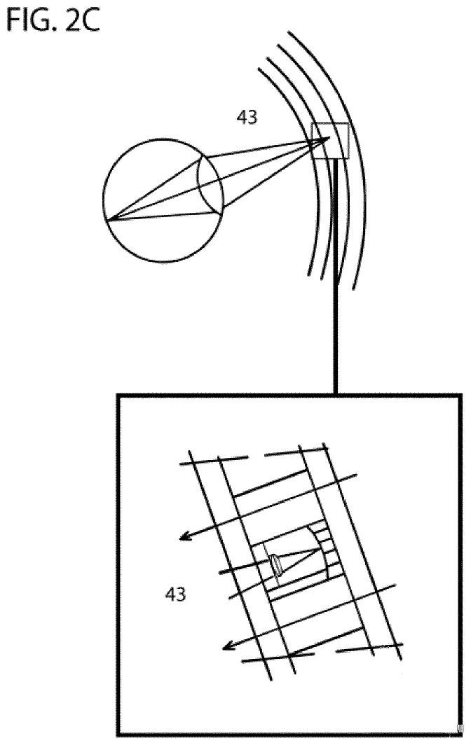

FIG. 2 presents a side view of a nanotechnology-embedded display of the eyeglasses configuration wherein the display array 42 is comprised of light beam emitters 43 and optional transparent, see-through regions 45 with a transformative diffusion layer 46 enabling the display to function as a watch face and an occlusion layer 44 enabling the display of present invention to function as an immersive, fully occluded virtual reality display or a see-through augmented reality display having the ability to varying the background contrast 44. The diffusive layer 43 may be between the light emitter 43 and the user's eye 92

FIG. 2A presents a side view of a direct center axis, illumination adjustment to a planar nanotechnology-embedded embodiment whereby the displayed image is shifted in response to the rotation of the eye. The insert shows the fine structure of the light emitting cell 43 having a controlled one, two or three dimensional emitter array to shift the perceived image.

FIG. 2B presents a side view of an off-center axis, illumination beam shift adjustment to a planar nanotechnology-embedded embodiment of the present invention.

FIG. 2C presents a side view of an off-center axis, illumination beam shift adjustment to a curved nanotechnology-embedded embodiment of the present invention wherein a primary shift may be unnecessary.

(Flexible Wraparound Display Tech)

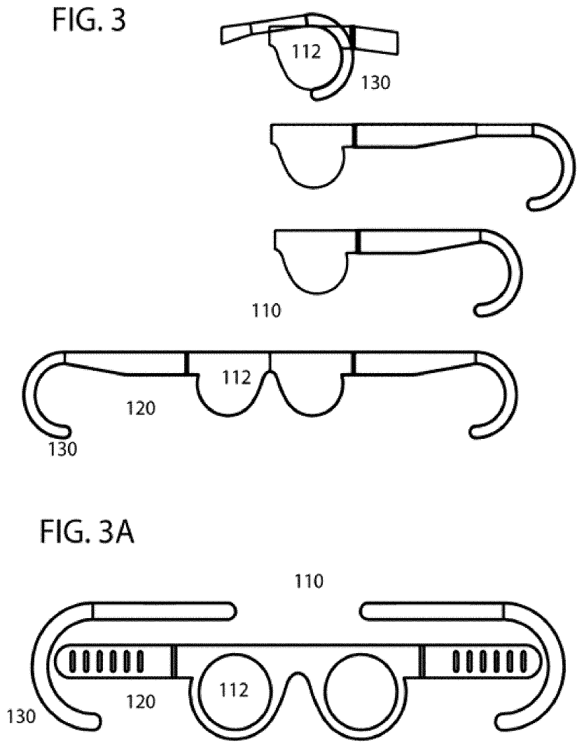

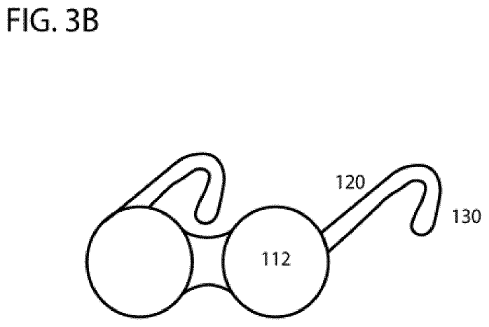

FIG. 3 shows a front view of flexible, transformative display device 100 in the four stages of transformation of the present invention. The flexible frame 110 holds the eyeglass lenses 112 and extends to include the proximal arms 120. Extendable distal arms and ear pieces 130 may be adjustably affixed to the proximal arms 120 and frame 110.

In a preferred embodiment, the display device 100 folds about the nose bridge and adjusts the arms 120 to enable the earpieces 130 to removably affixed to secure the device 100 to the user's wrist or any other object.

In a preferred embodiment, the device 100 has a positive curl imparted in the frame which causes the device to roll up in its natural state. This configuration enables the frame 110 to natural wrap around a user's wrist or be expanded to present sufficient curl force to stably affixed to a user's head, supported in part by the nose bridge.

FIG. 3A shows a transformative eyeglass to watch display embodiment with adjustable two-part arms of the present invention having a multiplicity of slots and an insertable section.

FIG. 3B shows a transformative eyeglass to watch display embodiment with elastic bridges and arms of the present invention.

### Virtual Reality, Augmented Reality Embodiment

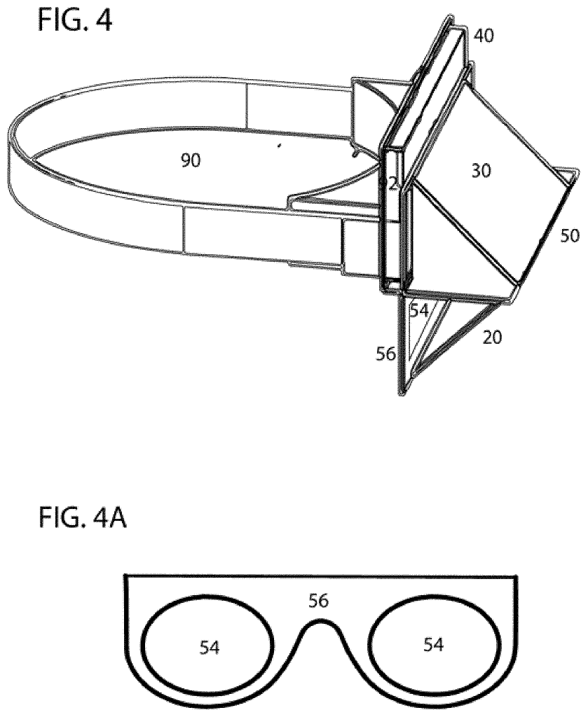

FIG. 4 shows a collapsible, fold-away, compactable, virtual reality lens embodiment of the present invention wherein virtual reality lens 54 may be movably affixed to a foldable VR Lens support 56.

In a preferred embodiment, the VR lenses 54 and support 56 may slide into a pocket behind the display 40 for storage or AR operation. In operation, the support 56 may removably affixed to the eye visor 20.

In another preferred embodiment, the VR lenses 54 and support 56 may movably and/or removably attached to the eye visor 20 and/or the device support 50. In operation for VR, the support 56 may removably but rigidly affixed to the eye visor 20 and the device support 50. When stored, the VR lenses 54 and support 56 may fold onto the eye visor 20 and both folded adjacent to the device support 50. In this configuration, the user's line-of-sight is direct.

FIG. 4A shows a user's front view of a preferred embodiment for the VR support 56 and VR lenses 54.

These preferred embodiments may incorporate any or all of the features disclosed in the parent applications including but not limited to U.S. patent application '044.

(Collapsible Head Strap Feature)

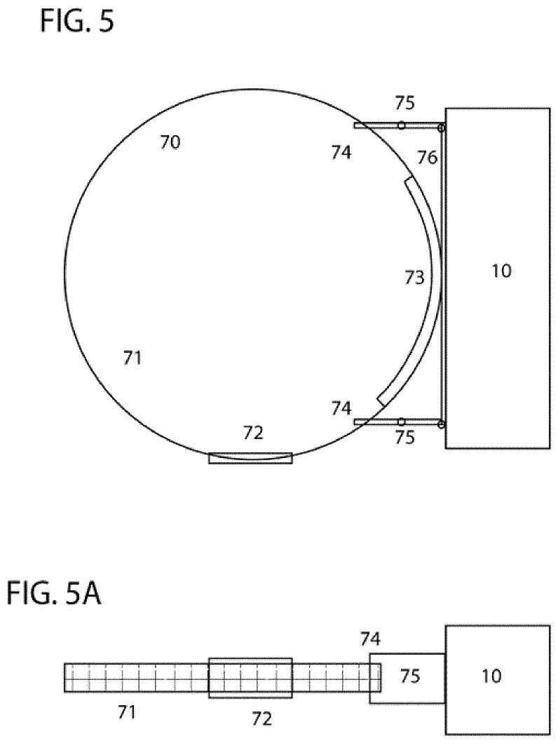

FIG. 5 shows a top view of a compact, collapsible, head strap feature of the present invention wherein the head strap apparatus is comprised of a strap 71 which may be made of any material, rigid, flexible or elastic; an strap fit adjustment 72 and an optional pad and/or shield 73 which may include but is not limited RF attenuation and thermal management. The headstrap apparatus may be securely fitted to the user's head or other object and is securely affixed to the device support frame 76 through attachment element 74 and attachment arm 75. The attachment element 74 may be adjustable and pivotable so as to maintain a secure and rigid attachment between the user and the headset apparatus 10.

The attachment arm 75 may collapsible, hinged, elastic or of other construction to enable a rigid and stiff connection between the head strap 71 and the headset apparatus 10.

FIG. 5A shows a side view of a compact, collapsible, head strap embodiment of the present invention. The attachment element 74 may be pivotable such that a counterbalancing force securely maintains the angular position of the headset apparatus 10.

((Game/Trade Show Variant of Ergo))

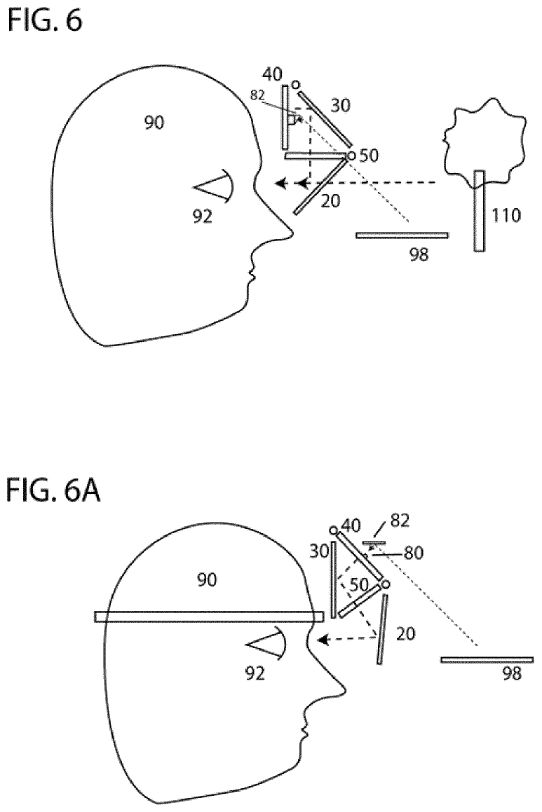

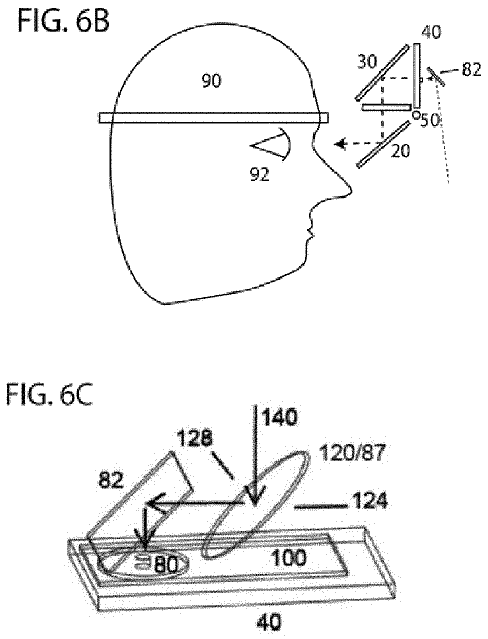

FIG. 6 shows a side view of a game or trade show, ergonomic, front facing display embodiment of the headset device 10 wherein the scene visualization means 80 includes a means 82 to redirect the field of view from the orthogonal to a proximal floor 98. In a preferred ergonomic embodiment shown in FIG. 6,

FIG. 6A shows a side view of a game or trade show ergonomic-rear camera embodiment of the present invention, wherein the scene visualization means includes a means to redirect the field of view from the orthogonal to an off-axis surface. The smartphone or display 40 faces the user 90 and the rear camera 80 is employed for external object recognition and other purposes.

FIG. 6B shows a side view of a game or trade show classical display embodiment of the present invention, wherein the scene visualization means includes a means to redirect the field of view from the orthogonal to an off-axis surface. The smartphone or display 40 faces the user 90 and is principally vertical in a manner compatible with current “Google Cardboard” applications. The rear camera 80 is employed for external object recognition and other purposes.

FIG. 6C shows a perspective view of a generalized scene, camera, or optical axis redirection component of the present invention, wherein the optical axis 140 reflects off the surface of mirror 120/87 to mirror 82 to sensors, projectors or scene visualization element means 80. One or more mirrors may rotate about any axis including axis 124 and be mounted upon optical redirection platform 100 which may rotate about the optical element 80. The platform 100 may be rotatable affixed to smartphone or module 40.

These preferred embodiments incorporate by reference any or all of the features disclosed in the parent applications including but not limited to U.S. patent application Ser. No. 16/190,044 and Provisional Patent Application No. 63/222,59.

(Name Tag Variant))

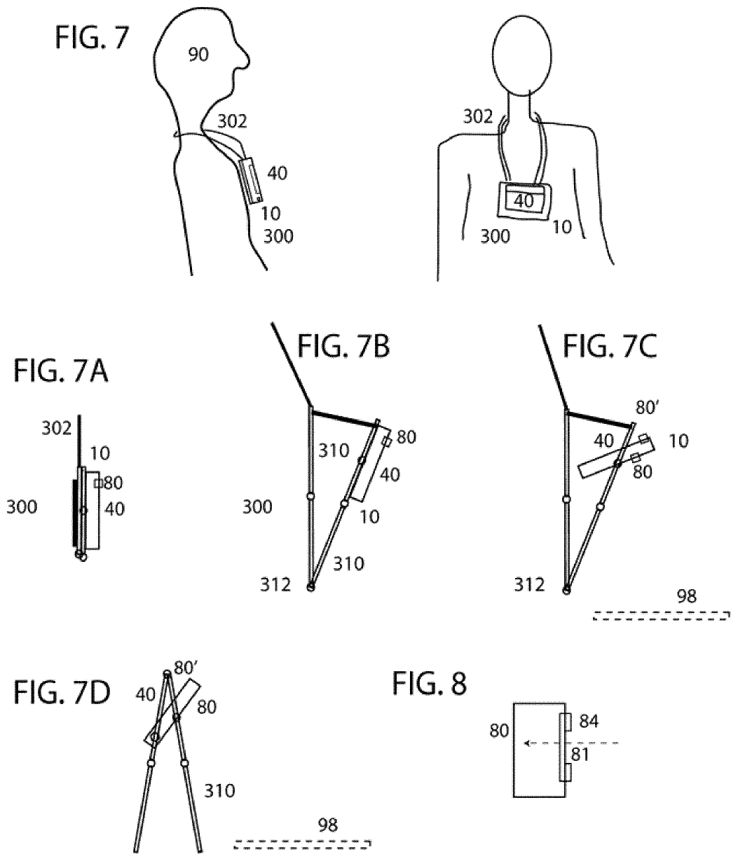

FIG. 7 shows side and front views of versatile Name-Game Tag embodiment 300 of the present invention wherein the compact headset apparatus 10, Smart Phone or other similar display device may be employed as a name-game tag device providing information on the display 40 to the audience, front camera (selfie) operation and monitoring and easy accessibility for the user 90. In its simplest form, the apparatus 300 is comprised of a paper holder for the smart phone and neck strap 302.

FIG. 7A shows the first of sequence of three, side views of a preferred construction of the versatile Game-Name Tag embodiment 300 of the present invention transforming from compact to object view. In operation, the apparatus 300 is comprised of a multiplicity of foldable members 310 which fold into a compact first configuration. The display 40 may face the user or the audience.

FIG. 78 shows a first expansion of the apparatus 300 wherein the display is facing the audience and the front ‘selfie’ camera is employed.

FIG. 7C shows a second expansion of the apparatus 300 wherein the display 40 is pivoted and facing the user; the rear camera 80 may be employed in game, information and other activities and the front ‘selfie’ camera may be employed for personal communication, recording or other activities.

FIG. 7D shows a tabletop preferred construction of the versatile Game-Trade Show Name Tag embodiment 300 wherein the members 310 form a self-supporting structure for any use including but not limited to monitoring activities, playing an augmented reality game with a designed board 98, or security applications.

(Diffusive Overlay for Optical Data Signal)

FIG. 8 shows a side view of the diffusive overlay 84 to an image camera 80 of the present invention for receiving an optical data signal. The parent applications describe an audience effects system which projects a static or dynamic optical pattern, picture, array, video or other Image upon an environment, audience, stage, building, room or other manifestation within which is embedded data or a data stream. The embedded data may be projected as invisible radiation (IR, UV, Acoustic, RF or other electromagnetic beam) or visible optical wavelengths wherein the temporal or spectral distribution encodes the data. Any protocol may be employed including but not limited to IRDA, Sony IR, RS232 and RLE.

The popular smart phone cameras may be employed in dual role: as a normal scene camera and as a data receiver. Normally, in order to receive a narrow data beam which may incident at any angle either the full frame must be analyzed. The process may be greatly simplified by dedicating part of the camera aperture, preferable in a plane of focus, to a diffusive or holographic filter which redirect part of the data beam to a dedicated region of the camera sensor. Thusly, the diffusive, translucent target in the field of view may be monitored for any beam characteristics (color, intensity and timing) of an external illuminating beam.

(Focal Distance by Divergence of the Emitted Beam of the Display Element)

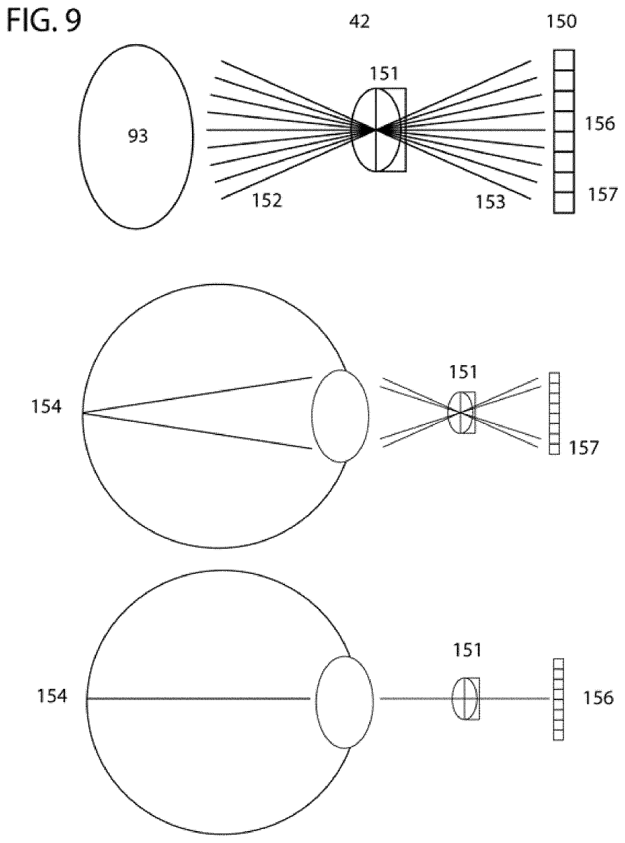

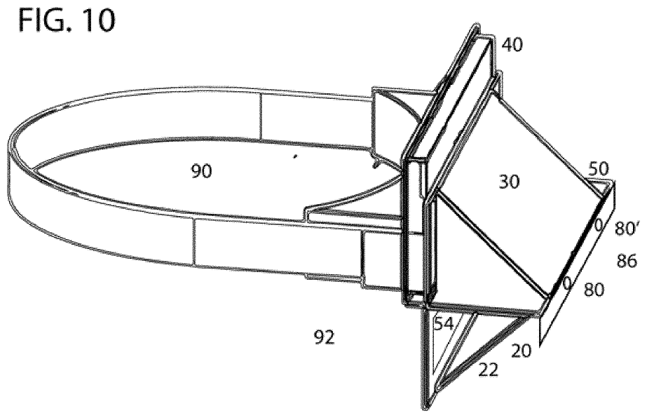

FIG. 9 shows a preferred method of and construction for varying the perceived focal distance of a display element of the present invention wherein the divergence of the light emissions from a given pixel 151 is adjusted by the simultaneous illumination of one or more light emitters 150, 156, 157 positioned about the principal optic axis of the emitter optics.

The perception of the distance of an object is determined by a number of factors including but not limited to the focal length of the lens of the eye; binocular convergence; image disparity; occlusion of or by other objects in the scene; relative size; relative or direction of the motion; color; and shading. The instantaneous focal length of the eye is in part determined by the divergence of the beam emitted from a resolvable, observable point source. The emitted beam may be of any form or combination including but not limited to conical or divaricated in one or multiple directions. For example, binocular emitter arrays, each pixel having a variable, horizontal divaricated form would enable the simultaneous projection of perceived focal distance (divergence), binocular convergence and image disparity.

FIG. 9A shows a preferred method of and construction for enabling a divergent array of light beams 157 to simulate a proximal display element. The response of the eye lens is enhancing by extinguishing the interior 156 nano-emitters 150.

FIG. 98 shows a preferred method of and construction for enabling a parallel beam of light 156 to simulate a distal display element.

(Construction Flip Up Eye-Optics)

FIG. 10 presents an interactive game-name embodiment which may be employed in manifestations of any sort and receive optical data signals of any sort having the flip-up eye visor 20 with optional VR lenses 54 and mixed-reality (MR) stereo cameras 80, 80′

((Ergo Name Tag Variant from App '044))

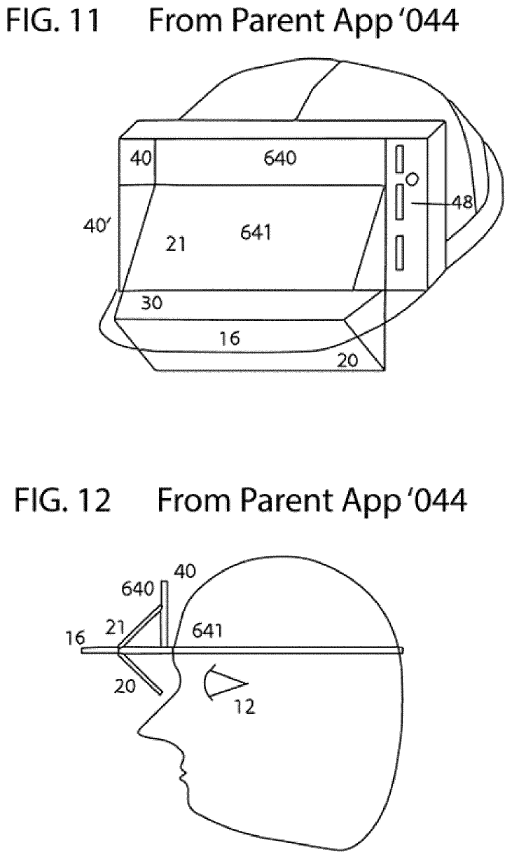

FIG. 11 shows a perspective view of a game or trade show embodiment of the present invention, wherein the display visualization means includes an external display area.

FIG. 12 shows a side view of a game or trade show embodiment of the present invention, wherein the display visualization means includes an external display area.

(Eye Sensors)

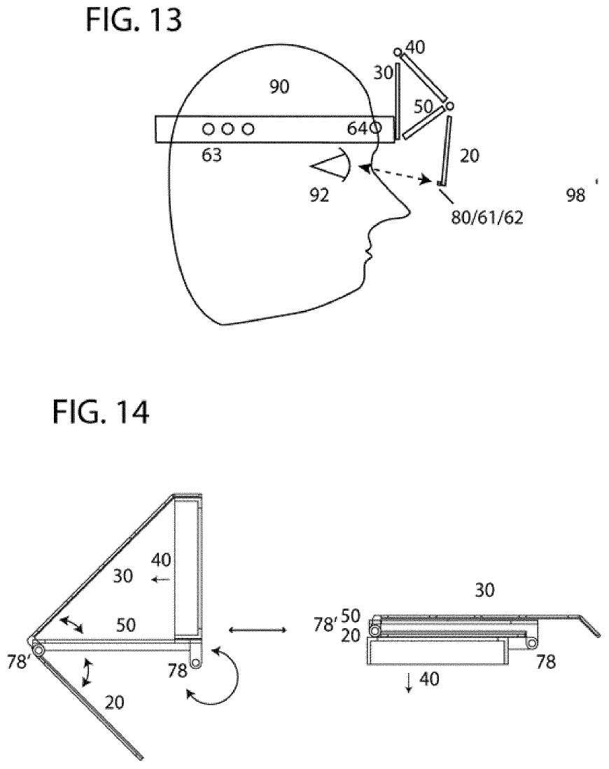

FIG. 13 presents an integration of multiplicity of sensors to monitor the physical and cognitive state of the user. Among, but not limited to, the sensors which may be integrated or removably attached are a eye state camera 80, illuminator 61, OCT, spectrometer or other eye or visual test apparatus 62; cognitive, neurophysiology or magno-electroneurophysiology sensors; and physiology sensors 64 such as body temperature, perspiration, heart rate, blood pressure and other parameters.

(Opera Designs)

FIG. 14 shows a side view of a clam-shell, foldable embodiment of the present invention wherein the eye visor pivots 78′ upward into a protected pocket in the lens support element 50 and the display (smart phone) 40 pivots 78 clockwise such that the display 40 remains visible (downward) to the user. The first mirror 30 pivots 78′ clockwise protecting the inner optical surface in a recess of the lens support element 50. A virtual reality lens and support (not shown) may pivot 78 inside of the display 40.

Alternative configurations may be employed including but not limited to a snap-out, sliding and fold from a front pivot 78′. Having thus described several aspects of at least one embodiment of this invention, it is to be appreciated that various alterations, modifications, and improvements will readily occur to those skilled in the art. Such alterations, modifications, and improvements are intended to be part of this disclosure and are intended to be within the spirit and scope of the invention.

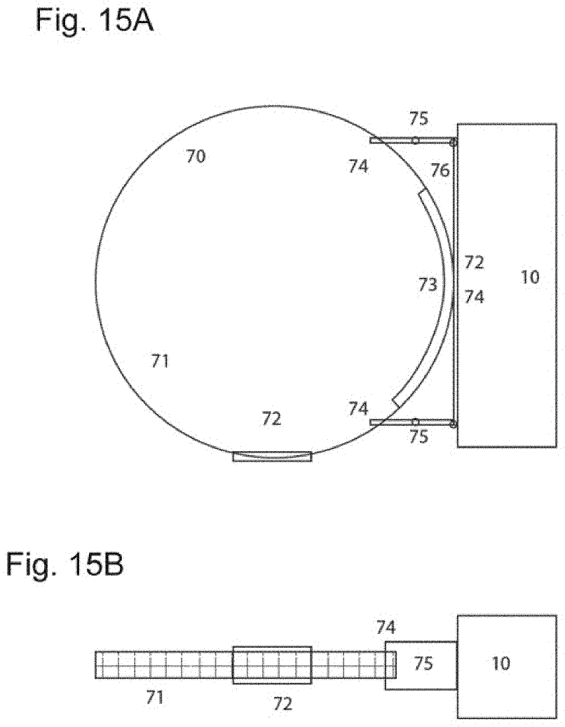

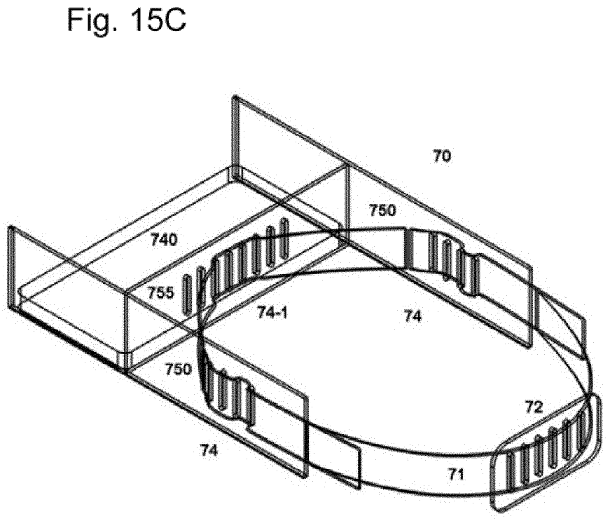

(Collapsible Head Strap Feature FIGS. 15-20)

FIGS. 15A-E shows a top view of a compact, collapsible, head strap feature of the present invention wherein the head strap apparatus is comprised of a strap 71 which may be made of any material, rigid, flexible or elastic; a strap length adjustment device 72; an optional comfort and safety device 73 which may be a foam comfort pad 73/730 having have spacers, patterns, cut-outs or special materials and composites to adsorb and dissipate body heat and sweat; and/or RF shield for the attenuation of RF in the direction of he forehead The headset comfort and safety device 73 may independent of the headstrap 71. The headstrap apparatus may be securely fitted to the user's head or other object and is secured to the device support frame 76, either static or dynamically through attachment element 74 and attachment headset arm 75 which may be rigid vertically while flexible horizontally.

The attachment element 74 may be adjustable, elastic and flexible so as to maintain a secure and rigid attachment between the user and the headset apparatus 10 at the forehead (72, 73, 74) while stabilizing the headset arm 75 attachment elements 74 proximal to the user's ears. The headstrap 71 may slide horizontally within the attachment device 74 or may be statically affixed to the headset 10 at one or more attachment elements 74, which may also integrate the adjustment device 72. In a preferred simplified embodiment the headstrap 71 is comprised to two sections, each of which is statically attached at adjacent forehead points, dynamically attached to the headset arm 75 attachment elements 74 and adjusted in length by the independent adjustment device 72.

The attachment arm 75 may collapsible, hinged, elastic or of other construction to enable a rigid and stiff connection between the head strap 71 and the headset apparatus 10.

FIG. 15B shows a side view of a compact, collapsible, head strap embodiment of the present invention. The attachment element 74 may be pivotable such that a counterbalancing force securely maintains the angular position of the headset apparatus 10.

FIG. 15C presents an isometric view of the present invention having a multiple slot 74 attachment and adjustment regions and elements 72. This preferred embodiment lends itself to fabrication from a flat sheet of material which may be comprised of but not limited to: paper, cardstock, cardboard, synthetic paper, plastics, composite woods, foam plastics, and composites etc.

This view of the present invention having a pliable inelastic or elastic headstrap 71, with or without a length adjustment 72, may be employed with zero, one or more fixed or sliding attachments 74 to the headset apparatus 10/70. In a preferred embodiment the headstrap 71 is immovably affixed to the headset 10/70 proximal to the forehead panel 74-1 and may have pass-through attachments 74 on the headset temple arms 75/750. The headstrap 71 may be adjustable at the attachment points 74 on the forehead element 755, the headset temple arms attachment 74; or an independent strap adjustment device 72. The headset temple arm 750 may be articulated at one or more points 76 enabling the headset arms 750 to fold within the outline forehead (device support frame) headset element 755.

In a preferred embodiment one or more regions 640, 48 of the Display Module/Smart Phone Screen 40 is visible and/or accessible for touch screen activation. The visual and touch characteristics be dynamically altered in response to any event, location, command, etc. In combination with the sensor input including spatial positioning and status, the region 640 may function as a show and/or user controller “pixel” as part of a large collection of the Receivers.

The Smartphone may be removed through any openings, folds or other constructions in any direction including but not limited to refastenable retaining flaps on the sides or the top comprising an integral or separate element.

inelastic or elastic headstrap 71, with or without a length adjustment 72, may be employed with zero, one or more fixed or sliding¬attachments 74 to the headset apparatus 10/70. In a preferred embodiment the headstrap 71 is immovably affixed to the headset 10/70 proximal to the forehead panel 74-1 and may have pass-through attachments 74 on the headset temple arms 75/750. The headstrap 71 may be adjustable at the attachment points 74 on the forehead element 755, the headset temple arms attachment 74; or an independent strap adjustment device 72. The headset temple arm 750 may be articulated at one or more points 76 enabling the headset arms 750 to fold within the outline forehead (device support frame) headset element 755.

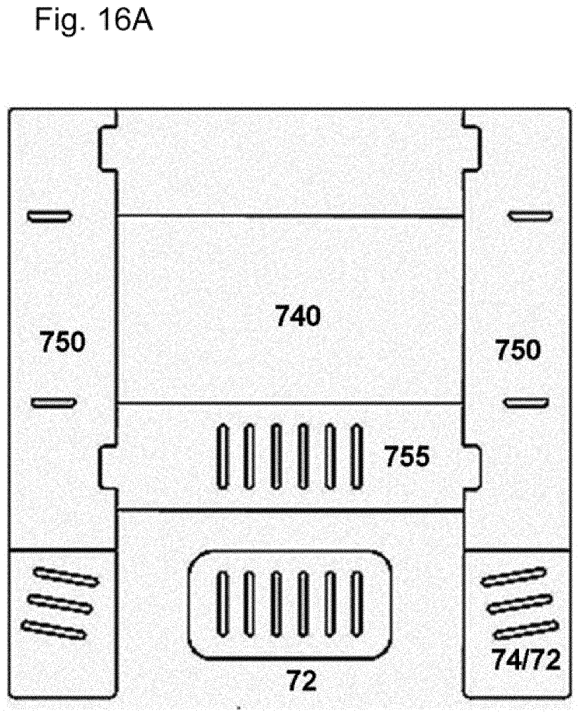

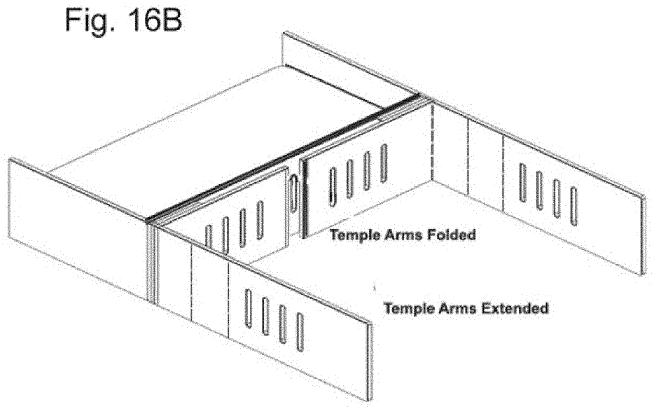

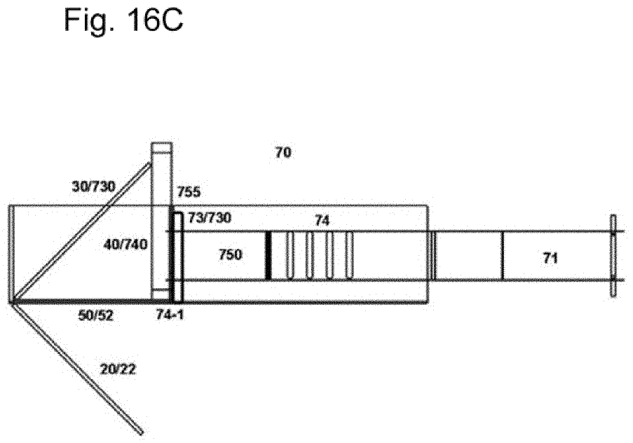

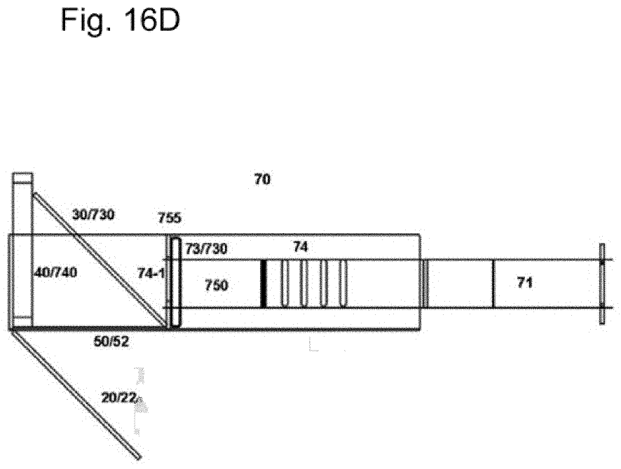

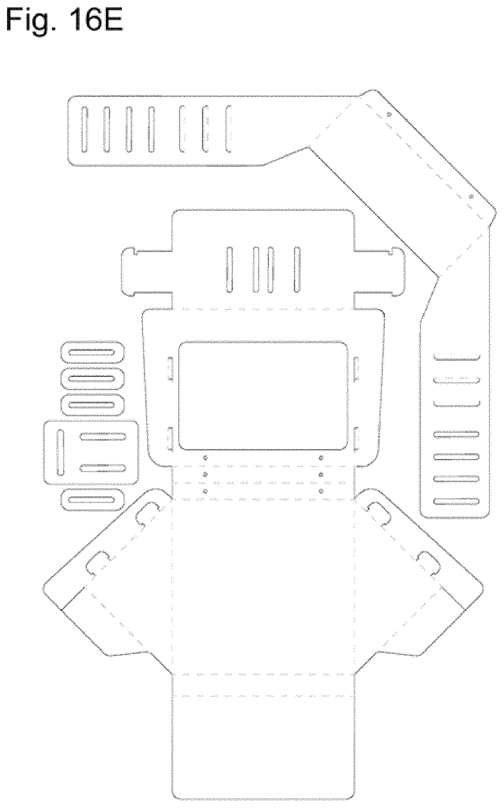

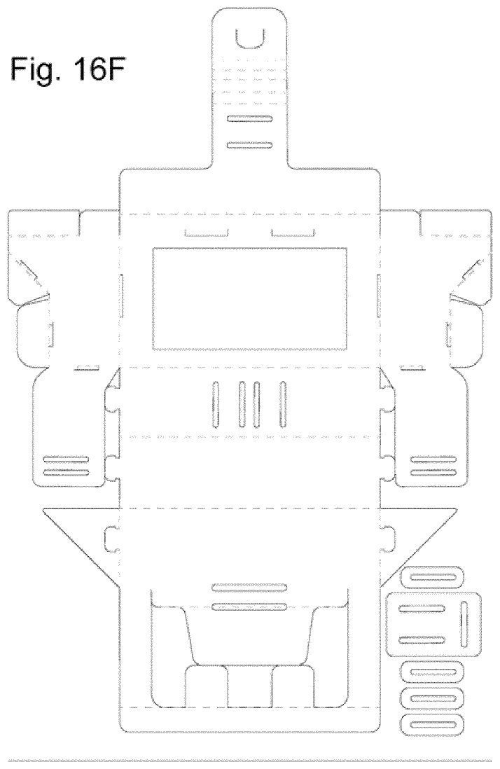

FIG. 16A shows a flat, foldable embodiment having a forehead contour region on the forehead panel 755 enabled by cuts and fold and/or cutouts 756 in the region of the fold-axis between the 740 phone/lens platform and 755 forehead panel. There may be multiple radiating cuts and creases enabling the resultant sections to fold towards the vertical and thereby further cushion the forehead.

FIG. 168 shows perspective and folded view of the flat, foldable embodiment having the temple arms in both the extended and stored folded positions.

FIG. 16C shows a side view of a game or trade show, ergonomic, front facing display embodiment of the headset device 10 wherein the scene visualization means 80 may include a camera or other optical sensor or matrix, an optical means 82 including but not limited to mirrors, prisms, holograms or meta materials, to redirect the field of view from the orthogonal to a proximal floor 98. References a preferred ergonomic embodiment shown in FIG. 67 in DS App 16190044. The Camera 82 may be outside the optical display system

This side view of a preferred ergonomic (ERGO) embodiment of the present invention having a pliable, inelastic or elastic, headstrap 71, with or without a length adjustment 72, may be employed with zero, one or more fixed or sliding attachment region 74 to the headset apparatus 10/70. In a preferred embodiment the headstrap 71 is immovably affixed to the headset 10/70 proximal to the forehead panel 74-1 and may have pass-through attachments 74 on the headset temple arms 75/750. The headstrap 71 may be adjustable at the attachment points on the forehead 74/740, the headset temple arms attachment 74; or an independent strap adjustment device 72/174. The headset temple arm 75o may be articulated at one or more points 76 enabling the headset arms 75 to forehead (device support frame) headset element 76.

A foam, cloth, or other material comfort element 73/730 may be provided at the forehead element 755. The comfort element 73/730 may be formed and attached in any manner including but not limited over the headstrap 71-attachment element 74 or as horizontal-array of spaced vertical cylinders affixed to surface of the forehead element 755, under or over the headstrap 71.

The ERGO embodiment is shown having the Display Module/SmartPhone 40/740 positioned proximal to the forehead 755, the first mirror 30/730 aligned to reflect the display image downwards to the lens element 50/52 and the eye reflector 20/22.

present a side view of preferred LEGACY embodiment of the present invention having the Display Module/SmartPhone 40/740 positioned proximal to the forehead 755, the first mirror 30/730 aligned to reflect the display image downwards to the lens element 50/52 and the eye reflector 20/22.

FIG. 16D present a side view of a preferred classical (DIRECT) embodiment of the present invention having the Display Module/SmartPhone 40/740 positioned proximal to the forehead 755, the first mirror 30/730 aligned to reflect the display image downwards to the lens element 50/52 and the eye reflector 20/22. The DIRECT embodiment is compatible with Goggle Cardboard and other Smartphone VR/AR headset designs.

FIG. 14 present a side view of preferred fully FOLDED OPERA embodiment of the present invention having the Display Module/SmartPhone 40/740 positioned proximal to the forehead 755, the first mirror 30/730 aligned to reflect the display image downwards to the lens element 50/52 and the eye reflector 20/22.

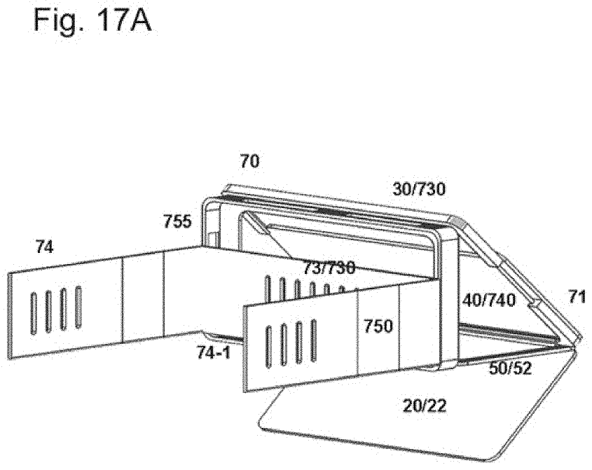

FIGS. 17A and 17B present perspective views of preferred Open OPERA WITH SLIDER HYPOTENUSE MIRROR embodiment of the present invention having the Display Module/SmartPhone 40/740 positioned proximal to the forehead 755, the first mirror 30/730 aligned to reflect the display image downwards to the lens element 50/52 and the eye reflector 20/22.

The First Mirror (Hypotenuse Mirror) may be comprised of a flexible material and unravel to increase the hypotenuse length; may be of accordion-design; and/or include a top and bottom slider elements 730 into which the mirror 30 slides. In the closed state, the sliders converge to reduce the overall hypotenuse length. In the open state, the sliders move outwards thus increasing the hypotenuse length. Side and supporting rails and inserts may be provided.

FIG. 17B present a side view of preferred Open OPERA WITH SLIDER HYPOTENUSE MIRROR embodiment of the present invention with folded headstrap and having the Display Module/SmartPhone 40/740 positioned proximal to the forehead 755, the first mirror 30/730 aligned to reflect the display image downwards to the lens element 50/52 and the eye reflector 20/22.

FIG. 18 shows a perspective view of a general, game or trade show, ergonomic, front facing display embodiment of the headset device 10 wherein the scene visualization means 80 may include a camera or other optical sensor or matrix and portions of the touchscreen display 40, 40′, 640, 48 may be visible and/or accessible. Identification, activation, marketing and may other applications may utilize this access.

FIG. 19 present a side view of preferred FOLDED OPERA w/ VERTICAL SLIDING CAMERA COVER embodiment of the present invention having the Display Module/SmartPhone 40/740 positioned proximal to the forehead 755, the first mirror 30/730 aligned to reflect the display image downwards to the lens element 50/52 and the eye reflector 20/22.

FIG. 20 present a side view of preferred FOLDED OPERA w/ HORIZONTAL SLIDING CAMERA COVER embodiment of the present invention having the Display Module/SmartPhone 40/740 positioned proximal to the forehead 755, the first mirror 30/730 aligned to reflect the display image downwards to the lens element 50/52 and the eye reflector 20/22.

Many additional preferred embodiments of the present invention, where the headstrap 71 may have fixed or sliding alignment/stabilizing slots at an intermediate strap 71/72.

The user's ear may support and stabilize the headset through the temple arm region 752 which may rest upon the user's ear. The headstrap 71 may constructed from two or three segments, and be adjustable at the attachment points on the forehead 73, 76; the headset temple arms attachment 74; or an independent strap adjustment device 174. Elements may included: Separate Arm-Forehead Strap, Single Adjustment, On headset arms, On forehead unit, Independent, Other, Dual Adjustments, On arm, On forehead, Independent, Other, Flow through, On arms slot, On forehead.

Various configurations may included: articulated Temple Arm-Fold Lines 76 lays flatter when folded. Articulation Fold Creases may be dimensioned such that the main part of the temple arm folds to cover 50% or less of the width of the forehead section; Foam, absorbent/dissipating materials, patterns, spacers from forehead pad; May be patterns with slots and/or attached from above or below the strap attachment region; Orientations—Strap Slot; Horizontal; Canted forward; Canted backward; Spaced above lens; Transmissive—Reflective Lens combination; ERGO, —Vertical Display Forward; LEGACY-Vertical Display Rear; Transformable Module Orientation; Headset may transform; Headset construction as a platform for independent Display Module.

Materials for construction of the headset may include: Paper, Synthetic, Plastic, Carbon fiber, Wood, Cloth or other.

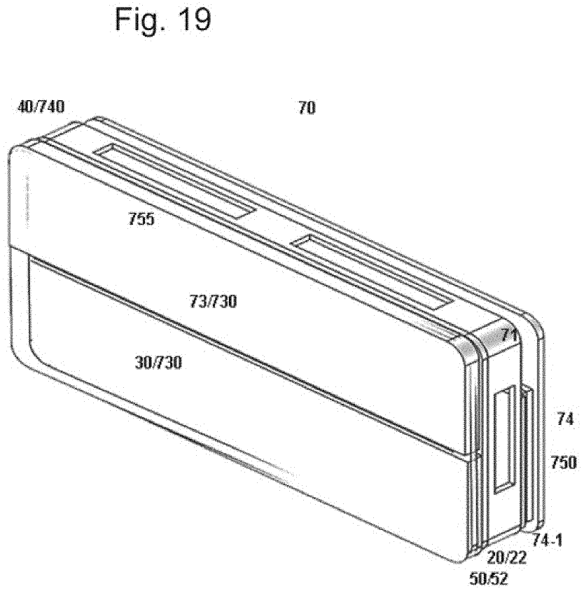

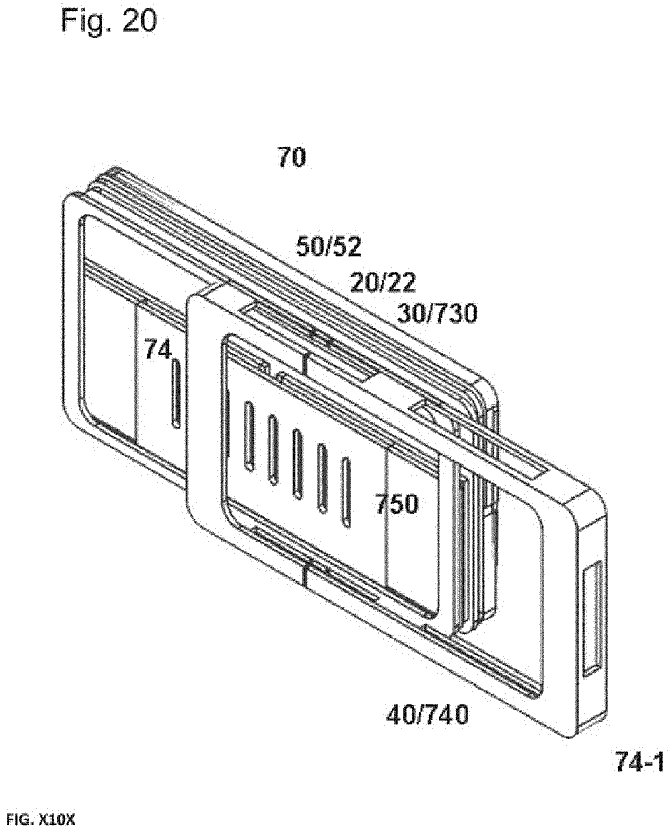

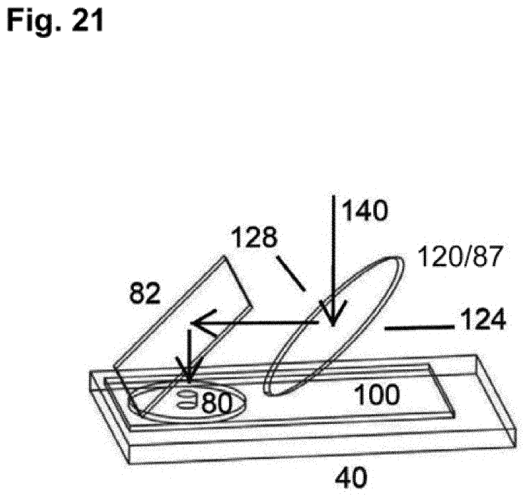

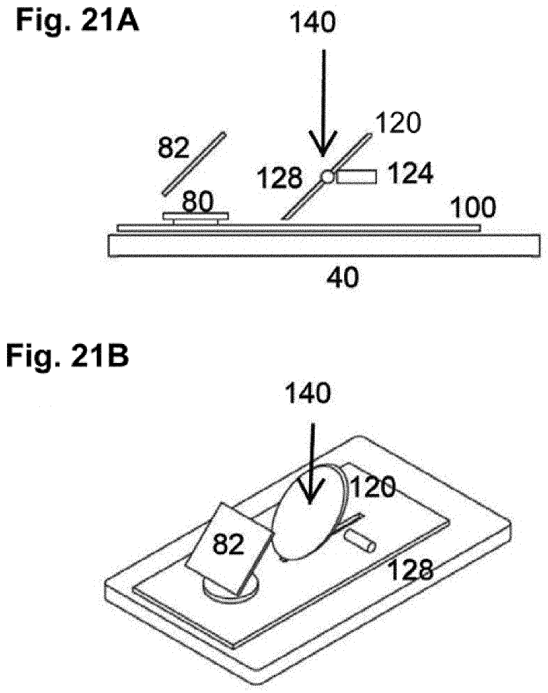

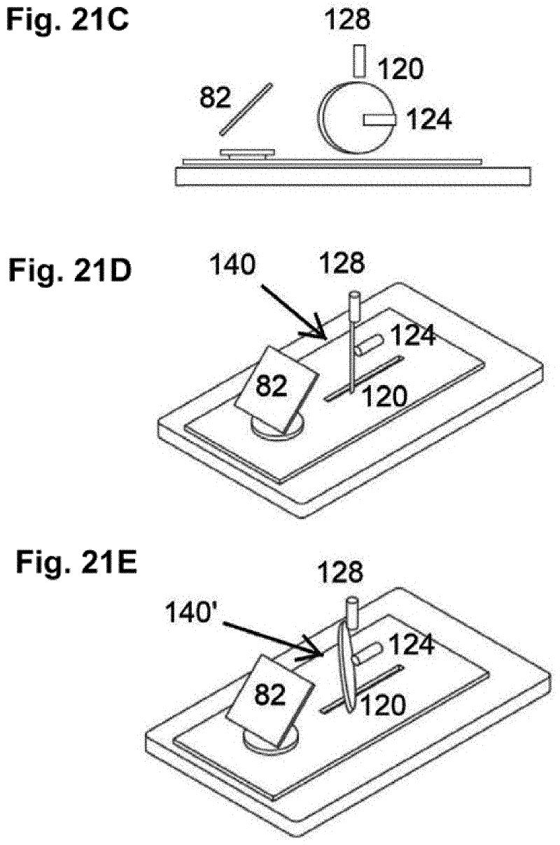

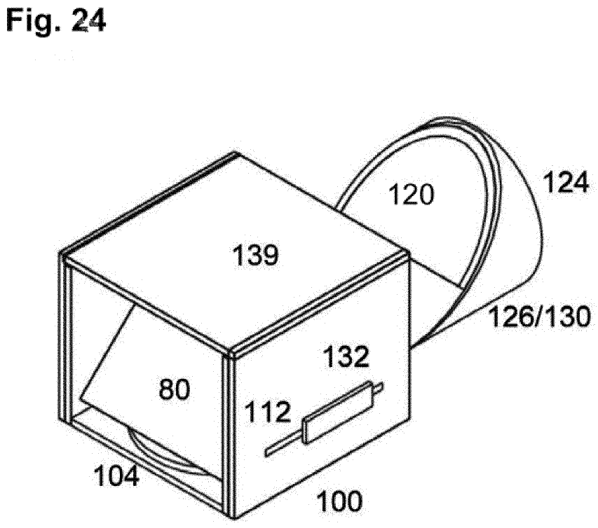

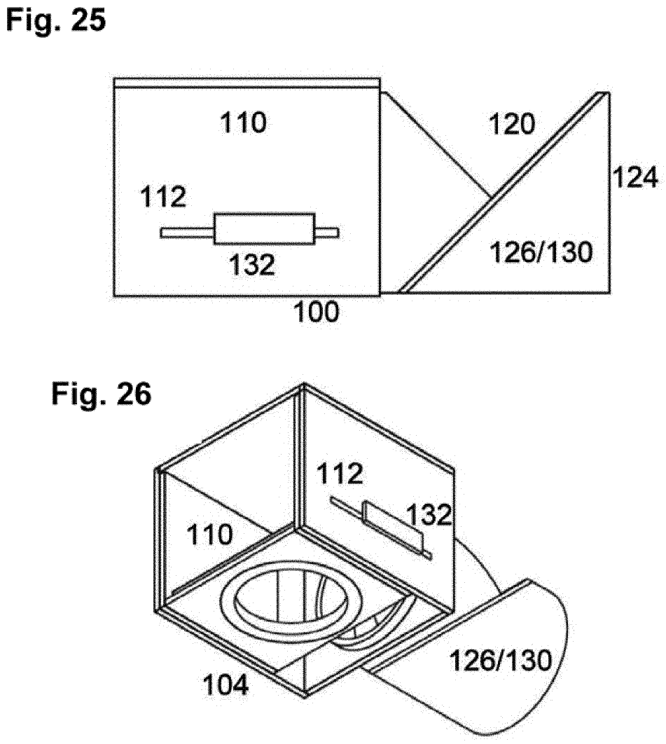

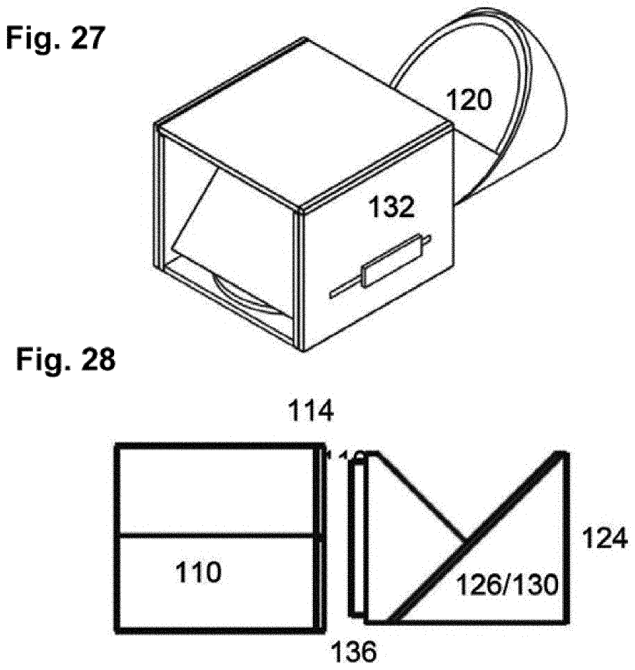

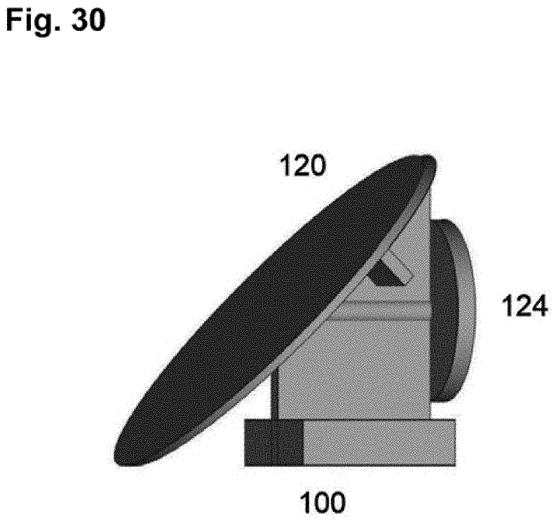

(Telescoping Optic Axis Redirection FIGS. 21-35)

Modern cell phones are increasingly employed as the display device for Head-mounted displays including, but not limited to, those designed by AR for Everyone and Google. The cell phone commonly has one or more integrated cameras, most often with in a fixed position with a principal optical axis orthogonal to and offset from the principal center of the phone. This arrangement presents a problem for the accurate registration of the camera image with the display image. Further the orthogonal principal axis presents a problem when the external object of interest in not in a comfortable or convenient location relative to the orientation of the user's head and HMO. Further, the distance between the principal optical axis and the phone center varies from phone to phone. Thus, an object of this invention is to vary the principal view axis of the camera, provides a system to align with the principal or designated axis of the phone and an optical system which folds compactly. This innovative and economical solution comprising a foldable, telescoping, rotatable optical system which may be better understood from the drawings and specification herein. While the camera 80 is shown and described as integrated into a contemporary “Smartphone” 40, the present invention may be applied to any camera or optical device of combination.

FIGS. 21A-E presents the basic concepts and elements of the present invention. The camera 80 integrated into the phone 40 views an external image 140 through the optical system comprised of first mirror 82 and a second mirror 87/120. The mirrors. 82, 87/120 are shown as flat, first surface mirrors oriented at 45 degrees, but these elements may be complex combinations of reflective, holographic, thin film and other optical elements including but not limited to holographic, prismatic and lens array elements and oriented at any angle which achieves the desired angle of incidence.

In normal operation the first mirror is fixed to a base 100 which may rotate about the axis of the camera 80. The second mirror rotates about axis 124 causing the incident view angle 140 to sweep a plane orthogonal to the main axis of the phone 40.

A second adjustment may be made by a transverse axis 138 introducing a change in the plane parallel to the main axis 40.

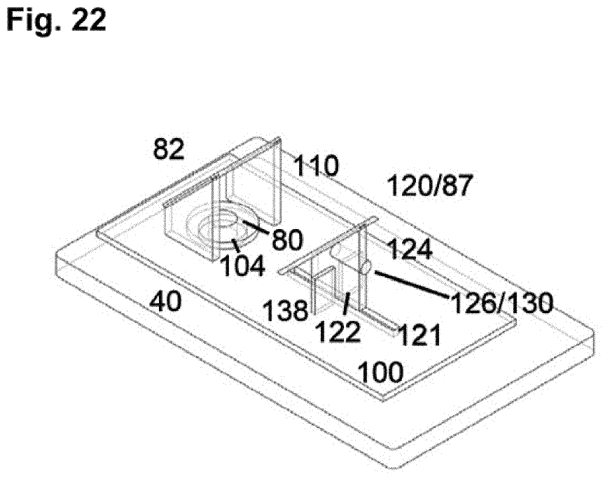

FIG. 22 presents a preferred foldable embodiment of the present invention wherein the 1st mirror 82 is supported by foldable supports 110. The 2^{nd} mirror is supported by foldable support 112 which may fold about the pivot axis 124 or a rotating cylinder 130 and knob lock

126. As shown in FIG. 22 only the top section of the 2^{nd} mirror 120 the top part of the mirror is attached to the support 112.

The 2^{nd} Mirror Assembly may telescope by sliding in telescoping slot 121. Further support may be provided by folding flaps 138.

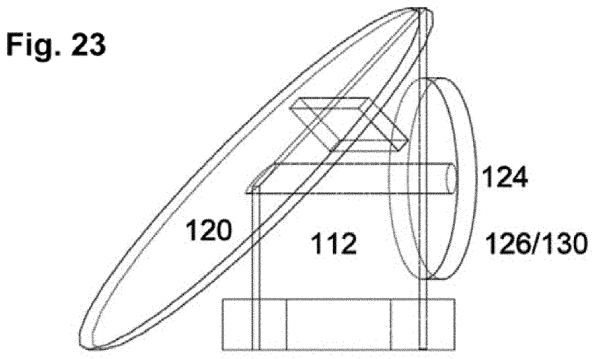

FIG. 23 presents another preferred embodiment of the present invention wherein the 2^{nd} Mirror Assembly 120/124/126/130 is rotatably attach a telescoping enclosure 139 by means of inner and outer race 114 and 136. In operation, the 2^{nd} Mirror Assembly 120-130 and 2^{nd} Mirror Telescoping Housing 139 vary the distance between said Mirrors 80 and 120 by actuating the outer 2^{nd} Mirror Telescoping Housing 139 maintained in registration by slot 112 and retaining element 132.

A retaining ring 100 affixed to the phone 40 which enables the base 100 to rotate about the camera axis 80 is shown.

FIGS. 24-27 present various enclosed and structured perspective views of general embodiment in FIG. 23.

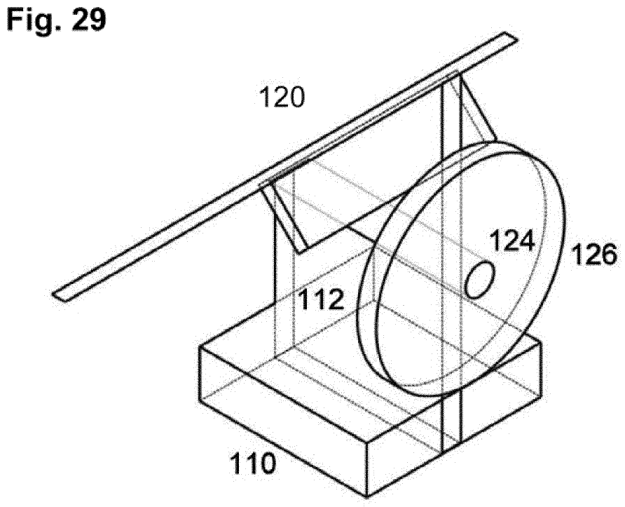

FIGS. 28, 29 and 30 present perspective views of alternative rotational assemblies of the present invention, where FIG. 28 is an inner and outer race 136, and FIGS. 29/30 are a central pivot 124 operated by a cylindrical disk 126.

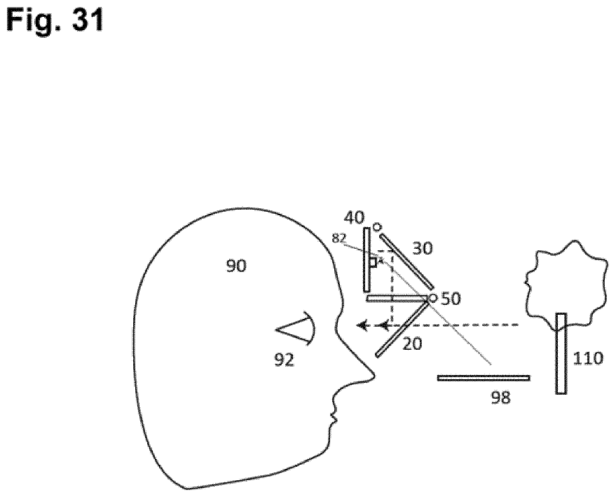

FIG. 31 shows a side view of a game or trade show ergonomic-rear camera embodiment of the present invention, wherein the scene visualization means includes a lateral means to redirect the field of view from the orthogonal to an off-axis surface. The smartphone or display 40 faces the user 90 and the rear camera 80 is employed for external object recognition and other purposes.

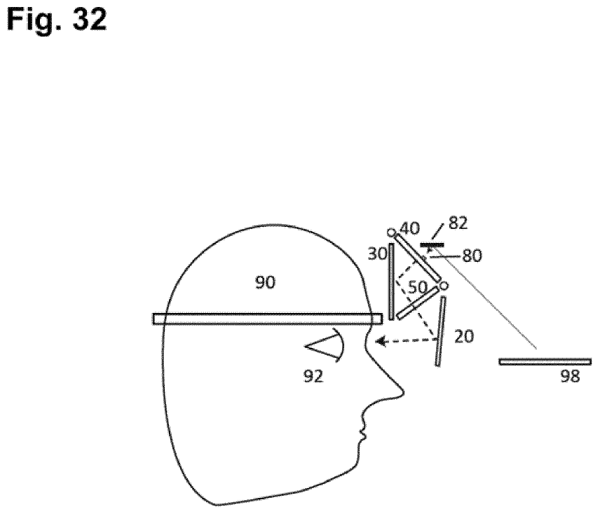

FIG. 32 shows a side view of a general, game or trade show oblique classical display embodiment of the present invention, wherein the scene visualization means includes a means to redirect the field of view from the orthogonal to an off-axis surface. The smartphone or display 40 faces the user 90, and may be incline about the vertical to reduce the moment of inertia relative to the forehead of the user 90. The main camera 80 may be redirect by mirror assembly 82. The inner or “selfie camera” may be employed to monitor the user's state 90 including but not limited to direction of vision, expression, physiology, response s to external or internally-generated stimuli.

FIG. 33 shows a side view of a general, game or trade show vertical classical display embodiment of the present invention, wherein the scene visualization means includes a means shown as mirror 82 to redirect the field of view from the orthogonal in an off-axis direction. This may be vertical in a manner compatible with current “Google Cardboard” applications.

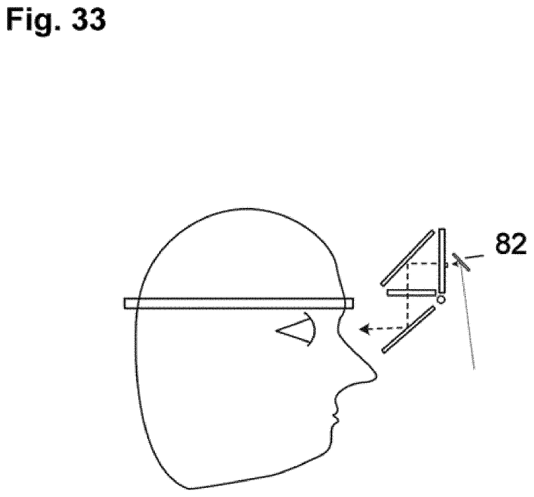

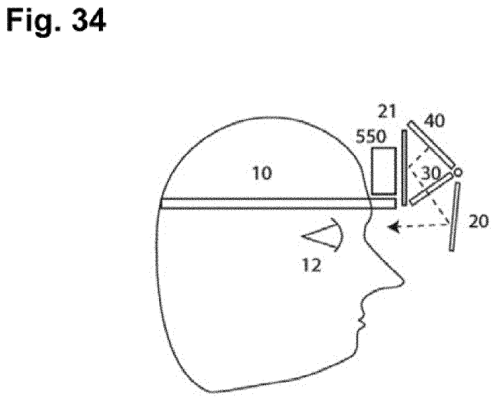

FIG. 34 shows a side view of a general oblique classical embodiment wherein the Smartphone or module 40 is offset vertically and the lens 30 is obliquely upward, and places the assembly on ergonomic head unit 550 which may include but is not limited to foam, air or other cushions; active cooling devices such as fans, circulators and solid state; This embodiment reduces the moment of inertia and required distance forward of the forehead.

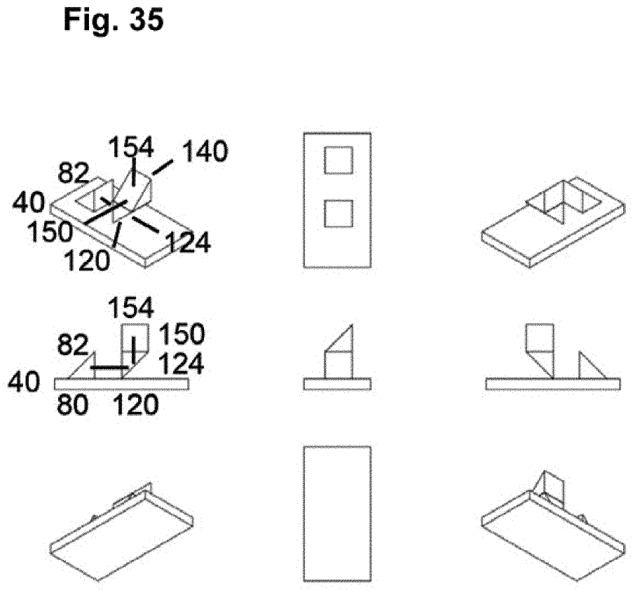

FIG. 35 shows various views of a preferred construction of the present invention employing simple cubic parts easily assembled from die-cut flat sheets.

FIGS. 36-37 show various views of a preferred construction of the present invention employing simple snap-together parts easily 3D printed.

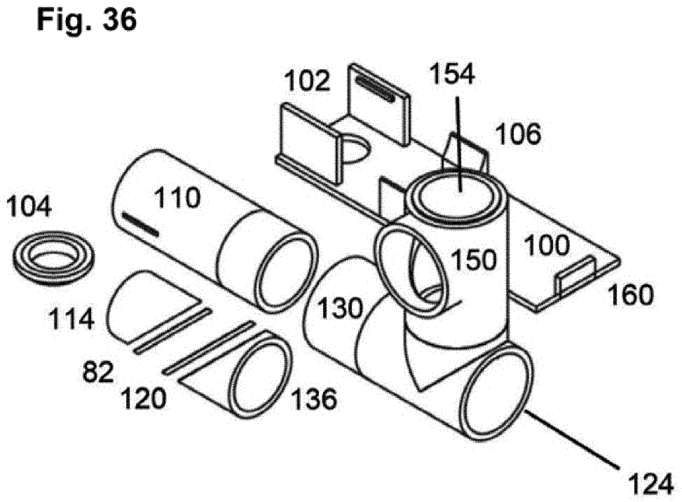

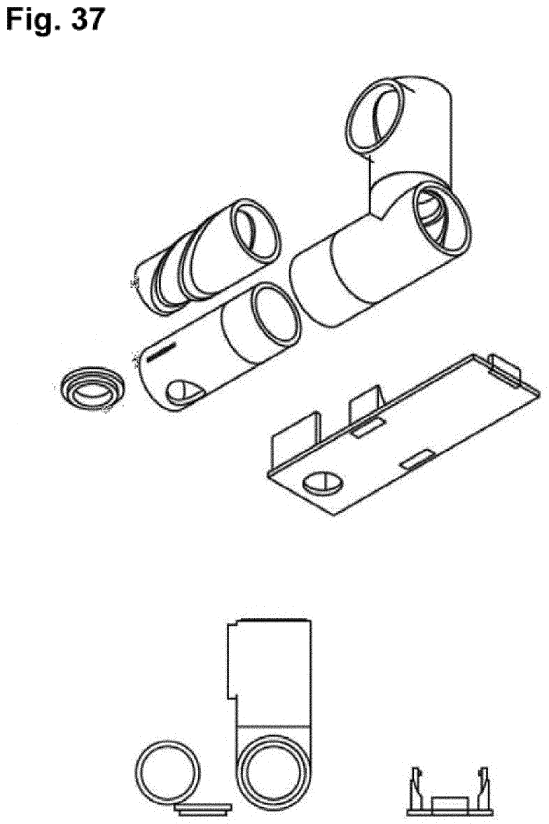

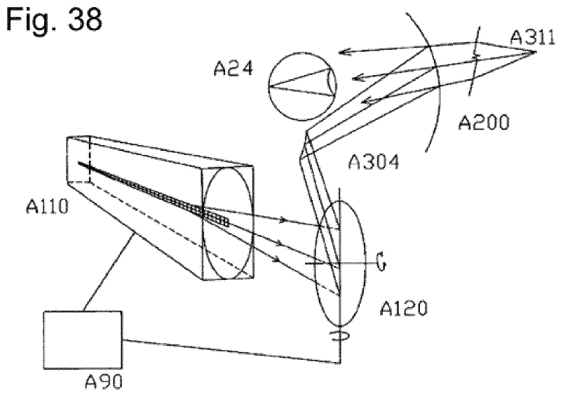

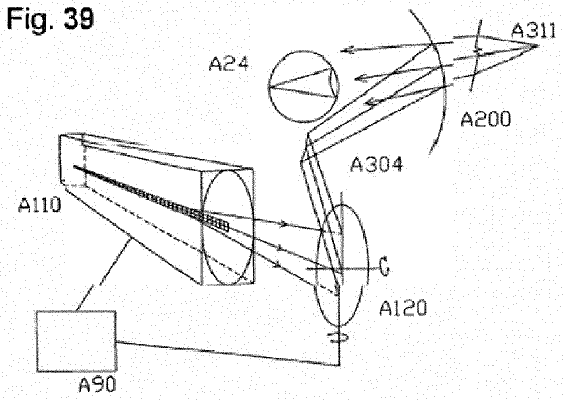

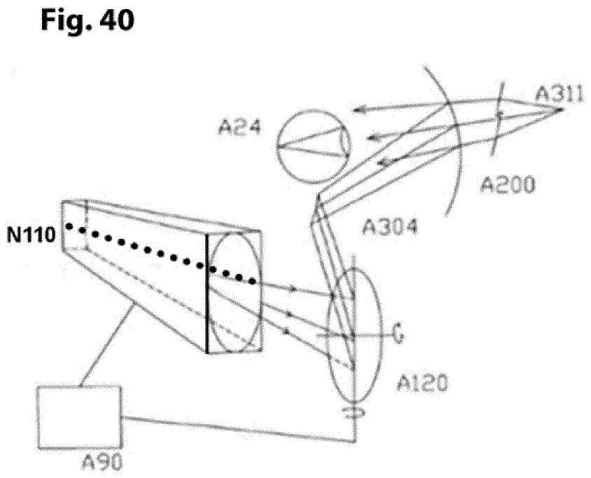

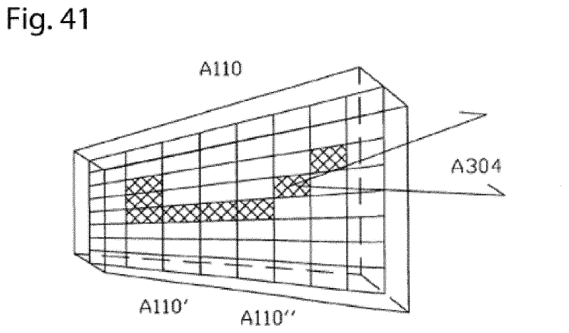

(Convergence & Accommodation) (FIGS. 38-41)

Human Eyes employ two complementary mechanisms for normal vision: Convergence—changing the angle of intersection of the principal optical axes of each eye, and Accommodation—changing the shape of the eye's lens and thereby it focal length. In our normal 3D environment, these mechanisms operate autonomically. (see Autonomic Control Of The Eye by David H. McDougal and Paul D. Gamlin, Compr Physiol. 2015 January; 5(1): 439-473, incorporated herein by reference.)1 1 https://www.ncbi.nlm.nih.gov/pmc/articles/PMC4919817/pdijnihrns795047.pdf

Current display technology simplifies our normal visual environment by presenting 3D environments as flat or monoscopic ‘representations’ (drawing, pictures, television, video, cinema) which eliminates both normal object convergence and accommodation—the viewer's eyes converge and focus at the same distance regardless of the “real world’ relationships between the objects in a “real” environment. One display technology which moves toward the “real world” is stereoscopy which presents slightly different images to each eye, thereby enabling an object-based convergence.

When the objects are within 5 meters of the observer, a significant conflict arises between the object-specific convergence and the fixed global accommodation. This conflict may produce eye strain, headaches; it reduces scene acquisition, interpretation, apprehension. This effect manifests in the physiological processes of the vision system, measurable at the eye, the optic nerve, the brain, the muscles, and the entire human body.

Object (or pixel)-specific accommodation requires that the divergent cone of light from each pixel of the display precisely converge at the proper ‘real world’ distance.

Various solutions have been proposed including phase and digital holography, and dynamic ‘lightfield’. The calculations requires and the apparatus to display or project are complex, artifactual, and incomplete.

The present invention discloses a novel method and improved apparatus for the display of the visual accommodation directly translates the depth coordinate, commonly designated the Z value in cartesian XYZ space to spatially-aligned display source emitter accurately displaced from the “zero” plane in the display optical system resulting in the proper true focal for each pixel at the observer's eye.

The apparatus in described herein.

The method includes the discrete element analysis of the image to be displayed relative to the principal axis and plane of the observer's eye lens wherein each elements distance, angular displacement, chromaticity and transparency is described. Any global or local coordinate system may be employed and encoded.

For the purpose of this generalized example, the method is presented using a cartesian coordinate model, wherein the image projected onto the observer's retina is described an X-Y (Z, C, T) array of discrete elements (pixels) where, by convention, X is lateral or horizontal and Y is vertical, and the intersection of the principal optical axis of the eye is represented by the array element (0,0). The focal distance of the image element (X,Y) is the value (Z); its color is value(s) (C); and the transparency (T). Numerous conventions exists for the description of color and transparency (RBGA, CYMK, CIE1931, etc.) and any one may be employed. For the purposes of this generalized example, the RBGA convention is employed.

Thus, the initial steps of this method is to described the image to be displayed as comprised of discrete elements in an array (X, Y, Z, R, B, G, A).

In three-dimensional computer graphics, the rendering process maps a complex 3D model to an 2D image (an X-Y array of pixels), where the sharpness may be degraded by accommodative realism, and the displayed color an integration of the color and transparency of all the 3D model point lying on the optical ray defined by the eye lens and the spatial coordinates.

Non-Linear Matrix L(z) for dynamic methods accounts for the non-linear focal response of the eye, time to focus, and relative brightness where the maximum brightness curve corresponds to the maximum pixel focus curve envelope.

### Resolvable Accommodation of Monoscopic Overlapping Displaced Objects

### Problem

Contemporary displays are viewed at a fixed distance from the observer. When a three-dimensional object is presented, the observer naturally analyzes the scene employing cues such as the distortion of the object, relative size, shading and shadows, occlusion, and with classic stereoscopy—image disparity. Visual accommodation—the variable focus length of the lens of the observers eye—is not employed.

In many real world situations, the inability of displays to enable visual accommodation limits visual cognition, situational awareness, response time and accuracy. In the case where two objects are co-axially located at different distances along a principal ray from an observer and entirely overlap, the objects will appear fused when rendered for a existing monoscopic or stereoscopic displays.

### Solution

The present invention presents a novel method and apparatus which enables the accurate display of high resolution visual accommodation of a scene co-axially positioned virtual objects with a minimum of computer graphics processing.

### Definitions

Scene—the real world, virtual CGI or other data set containing the images, objects to be displayed.

Trixel—3D pixel or element in a n-dimensional matrix mapping of 3D spatial coordinates generally described in cartesian systems as X, Y, Z, horizontal, vertical, depth, respectively.

Trixel Attribute—Each 3D spatial coordinate may associate any number of attributes in the data set including but not limited to RGB color, Alpha opacity, reflectivity, emissivity, etc.

Rendered Matrix—refers to a 2D spatial coordinate data set with a z-depth attribute for each 2D spatial coordinate matrix and additional optional attributes which represents a monoscopic view of the scene viewed by each eye of the observer.

Critical Attribute—Target of Interest Occluded but exhibiting initiating behavior triggers the Critical Modalities—reduction of the opacity of the proximal, alternating frames (proximal, distal), symbolic overlays, etc.

Display Queue—refers to the sequence and timing of Display Projection elements to activate based on the Display Projection Device (3D Volume, 2D Pixel Matrix, 1D Pixel Matrix, etc.)

Display Projection Device—refer to any device which creates a visual image viewable by the observer which includes but is not limited to screen television technology, light emitting pixels (LED, OLED, uLED, LCD, FLCD, DMD, DLP, Laser). A preferred embodiment is a 1D or 2D matrix of LEPs arranged parallel or obliquely to the principle optic axis of the System such that the LEP at the proximal side are optically (measured along the principal optic axis) closer to the observer's eye than the distal LEPs at the distal side.

The method comprises the following steps:

Create a data set which encodes the Scene with 3D spatial coordinate applied to Scene elements—objects, surfaces, etc.—computable as Trixels and optional Attributes. Contemporary 3D CGI programs generally encode object-based data which reduces the size of the data. Object-based data sets generally contain object descriptions such as: Object 1—Cube, Edge Length, Orientation, Center position and Attributes rather than an 3D coordinate ordered list of the attribute of each element.

Analyze the Scene and compute the Rendered Matrix. The novel Render Computation includes all distal Trixels occluded by more proximal Trixels along the principal axis of the observer's eye (monoscopic view) but visible in the peripheral rays (marginal, chief meridional ray, etc)

Compute the display queue based on the Image Projection Element employed. Derive the monoscopic renderings with pixel depth data and high acuity visibility/surround acuity (points visible in only one view pair).

Test for Critical Attribute behaviors

Transfer control data to Display Device and Run.

Que for projection (mode based on device—cubic, linear, oblique

Project synchronously with at least one axis having focusable arc.

(Sensors and Inducers Affixed to the Present Invention)

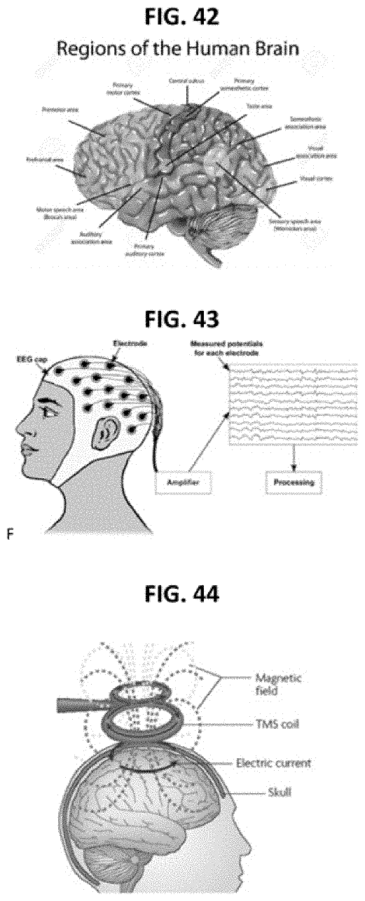

FIGS. 42-44 show examples of the regions of the human brain, the localization of the signals from events and stimuli, real, virtual and augmented, by electroencephalography, and the induction of stimulation by transcranial magnetic stimulation. These technologies may be integrated with the present invention, including but not limited to specialized receivers and inducers incorporated in the design of the elements of the headset and applications.

(Smartphone Camera Auxillary Input System Using Telescoping Optics) (FIG. 44)

Fig. The present invention discloses a compact, inexpensive, auxiliary, optical input technology for camera-based system 400 which does not require RF comm (Wifi, Bluetooth, 5G, etc) bandwidth, pairing or other registration. The optical input system 400 comprises a camera-based device such as but not limited to a SmartPhone 402 having a camera or light sensor input 404; and an independent, removably-attachable Auxillary Input Unit 410 comprises a black box controller 412; one or more data signal receiver elements 414; one or more output light elements 416 which may be modulators or sources (such as lasers, LEDs, LCDs, ODs, etc.) driven by the black box controller 412; and an optical combiner 430 such that the output from the output light element(s) 420 combines with the normal field of view 444 of the camera system 442.

In a preferred embodiment of operation, the black box 412 comprises an IR optical receiver (414) connected to processing unit 416 which transforms the data received in an encoded, infrared data signal (418) broadcast from a sender (450) into one or more changes in the static or temporal brightness or chromaticity of the output light elements 416. These changes are recognized by the camera and interpreted according to the commands of the software.

The data signal receiver elements 414 may be fixed or moving, point or rotated in any direction relative the host Smartphone 402.

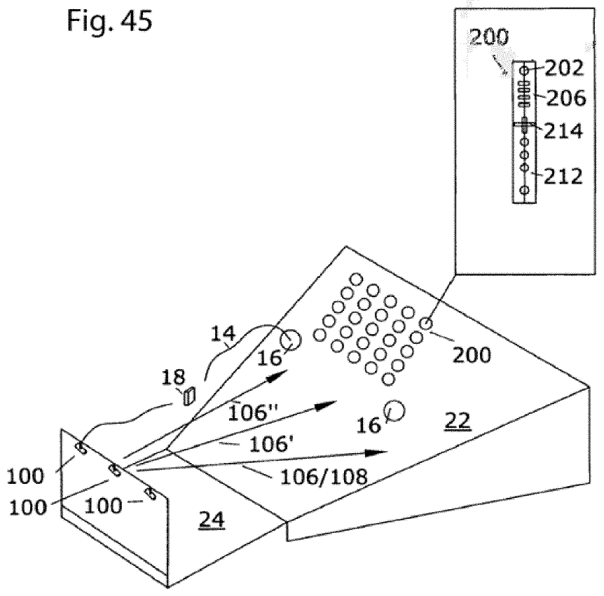

Audience Effects and Manifestions (FIGS. 45-48B)

The Experimental Reality System taught in the present invention incorporates by reference this Applicant's earlier applications and patents, including pending application Ser. No. 13/294,011. The System may be applied to audience effects, manifestations, or any distributed control application where simultaneously communication to a multiplicity of specific locations or radial directions from a signal emitter is advantageous.

In a simple embodiment, the System is comprised of a data source such as a stage projection/lighting control board, one or more image projectors such as the DIP series based on the Texas Instrument DIP® x-y matrix moving mirror shutter chip, and an audience receiver unit having a photonic receiver such as Smartphone camera. The communication transmission may be encoded in the modulation of one or more photonic wavelengths using known or custom electromagnetic (optical, RF, etc.) communication protocols

Data Source may be any device or storage media including but not limited to an entertainment media or light board, media server, DVD or any player, computer, smartphone, or integrated input device such as but not limited to a handheld baton, wand, wrist or body sensor,

Data set may be any digital and analog data describing any effects including but not limited to any visual data 2D or 3D picture, drawing, video, cartoon, art or abstract presentation; any audio data; any positional data; any motion data; or any other effects data (scent, vibration, stiffness, humidity, etc).

The communications system may be any device with transmits or projects the data set between the data source and receiver module including but not limited to a electromagnetic projector at any single or multiple wavelength(s) such as a UV, visible or IR projector, RF or ultrasound transmitter or audio speakers.

The receiver and effects module may be any device with receives the data set and produces the related effect.

Raving a receiver module comprising a smartphone or augmented reality headset.

In accordance with a first aspect of the present invention, a projection system is provided. The projection system is for providing a distributed effects within a location. The projection system comprises a data source, a projector and a plurality of receiving units distributed within the location. The data source for generating a plurality of data sets of associated effects data and spatial coordinate data. The projector is in communication with the data source for receiving the data sets therefrom. It comprises a signal generating module for generating a plurality of electromagnetic signals, each one of the electromagnetic signals being representative of the effects data from one of the data sets. The projector also includes a projecting module for projecting each of the electromagnetic signals towards a target location within the location. Each target location corresponds to the spatial coordinate expressed by each one of the receiving units depending at least in part on the target location at which the one receiving unit resides when the electromagnetic signal is received.

The plurality of receiving units is distributed within the location. Each receiving unit is provided with a receiver for receiving one of the electromagnetic signals when the receiving unit is positioned in the corresponding target location. Each of the receiving units is also adapted to perform a change of state in response to the effects data. In accordance with another aspects of the invention, there is provided a projector for providing a distributed effect within a location through a plurality of receiving units. The receiving units are adapted to perform a change of state and are positioned at target locations within the location. The distributed effect is based on a plurality of data sets of associated effects data and spatial coordinate data. The projector first includes a signal generating module for generating a plurality of electromagnetic signals, and encoding each one of these electromagnetic signals with the effects data from one of the data sets. Encoded electromagnetic signals are thereby obtained. The projector further includes a projecting module for projecting each of the encoded electromagnetic signals towards one of the target locations within the location corresponding to the spatial coordinate data associated to the effects data encoded within the electromagnetic signal. Preferably, the projector is provided with an encoder and the receiving units are each provided with a decoder. Preferably, the encoder is a modulator and the decoders are demodulators. Still preferably, the effects data is representative of a video stream and the receiving elements are provided with LEDs. In accordance with yet another aspect of the present invention, a method is provided. The method comprises the steps of: a) generating a plurality of data sets of associated effects data and spatial coordinate data; b) generating a plurality of electromagnetic signals, each one of the electromagnetic signals being representative of the effects data from one of the data sets; c) projecting each of the electromagnetic signals towards a target location within the location corresponding to the spatial coordinate data associated with the effects data transmitted by the electromagnetic signal; d) distributing a plurality of receiving units within the location; and e) at each of the target locations where one of the receiving unit is positioned: i) receiving the corresponding electromagnetic signal; and ii) changing a state of said receiving unit in response to the effects data. Advantageously, the present invention allows updating individually a plurality of receiving units with a wireless technology in order to create a effect, for example a visual animation. Embodiments of the invention may advantageously provide systems for displaying or animating elements by controlling or animating them from at least one centralized source. Control of these elements in function of their locations within a given space may also be provided, while not limiting their displacement within this space. Embodiments may also provide the capability of wirelessly updating the modular elements dispersed within the given space.

In accordance with a first aspect thereof, the present invention generally concerns a projecting system for creating am effect using a projector and several receiving units distributed within a given location. Electromagnetic signals are sent by the projector and may vary in function of specific locations targeted by the projector. In other words, receiving units located within a target location of the location will receive specific electromagnetic signals. These signals will include a effects data, instructing the receiving element on a change of state they need to perform. The change of state can be for example a change of color. The combined effect of the receiving units will provide a effect, each unit displaying a given state according to its location. The expression “effect” is used herein to refer to any physical phenomena which could take place within the location. In the illustrated embodiments, the effect is a visual animation, such as a change in color, video, or simply the presence or absence of light or an image. The present invention is however not limited to visual animations and could be used to provide other types of effects such as sound, shape or odor. The location could be embodied by any physical space in which the effect takes place. Examples of such locations are infinite: the architectural surface of a public space, a theatre, a hall, a museum, a field, a forest, a city street or even the ocean or the sky. The location need not be bound by physical structures and may only be limited by the range of propagation of the electromagnetic signals generated by the system, as will be explained in detail further below. The receiving units can be dispersed in any appropriate manner within the location. At any given time, the receiving unit may define a 2D or a 3D effect. The effect within the location may be fixed for any given period of time, or dynamic, changing in real-time or being perceived to do so. The distribution of receiving elements within the location may also be either fixed or dynamic, as will be apparent from the examples given further below. Referring to FIG. 1, a projection system 10 according to an embodiment of the invention is shown. The projection system 10 includes a data source 18, a comm projector 100 and a plurality of receiving units 200. In the illustrated example the plurality of receiving units 200 are provided with LEDs and together form the effect.

Components of projection systems according to embodiments of the invention will be described in the following section.

Data Source

Referring to FIG. 45, the data source 18 can be a computer, a data server or any type of device provided with memory and a processor able to store and transmit data to the comm projector 100. In operation, the data source 18 generates a plurality of data sets. The data sets generated by the data source 18 can include real-time state changes, cues or sequences of state changes to be executed by receiving units 200 located at a specific target location within the location. Each data set generated includes at least effects data associated with spatial coordinate data. Of course, the data sets may include further information, such as headers including information which identifies the information that follows, block of bytes with additional data and/or instructions, as well as trailers, for confirming the accuracy and stats of the data transmitted. As an example only, the data set illustrated takes the form of a data structure, in which part of the payload includes effects data while another part of the payload includes spatial coordinate data. Streams of data sets can take the form of an array, a table, a queue or a matrix containing numerous data structures.

“State”

The term “state” refers to a mode or a condition which can be displayed or expressed by a receiving unit. For example, a state can take the form of a visual effect, such as a color, a level of intensity and/or opacity. The state can also relate to a sound, an odor or a shape. It can be a sequence of state changes in time. For example, the effects data can be representative of a video stream, the distributed effect displayed by the receiving units 200 being a video, each receiving unit 200 thus becoming a pixel within a giant screen formed by the plurality of units 210. In order for the comm projector 100 to address specific receiving units 200 within the plurality of units, the effects data is associated with spatial coordinate data. The term spatial coordinate refers to a coordinate which may take various forms such as for example a position in an array of data, a location within a table, the position of a switch in a matrix addresser, a physical location, etc. Projector Now with reference to FIGS. X to X, different embodiments of the comm projector 100 are shown. A comm projector 100 can be any device able to project directional electromagnetic signals. It can be fixed or mobile, and a comm projection system according to the present invention can include one or several projectors. The comm projector 100 is in communication with the data source 18 and receives the data sets therefrom. While in FIG. 1 the data server 18 is shown apart from the comm projector 100, it can be considered to include the data source 18 within the comm projector 100. In either case, the connection between the data source 18 and the comm projector 100 allowing communication therebetween can be either a wired link or a wireless link. The comm projector 100 includes a signal generating module and a projecting module. The projector first includes a signal generating module for generating electromagnetic signals including of the effects data contained in the data sets received. In other words, each electromagnetic signal 26 generated by the module is representative of a specific effects data contained in a corresponding data set.

In a preferred embodiment, the electromagnetic signals have a wavelength within the infrared spectrum. Other wavelengths may be considered without departing from the scope of the present invention. The signal generating module preferably includes one or more light emitters. Each light emitter generates corresponding electromagnetic signals. The wavelength of the electromagnetic signals may be in the infrared, the visible or the ultraviolet spectrum, and the signal generating module can include light emitters generating electromagnetic signals at different wavelengths. The electromagnetic signals may be monochromatic or quasi-monochromatic or have a more complex spectrum. For example, the light emitters may be embodied by lamps, lasers, LEDs or any other device apt to generate light having the desired wavelength or spectrum. Referring more specifically in particular embodiments of the invention, the signal generating module 24 may include an encoder for encoding each electromagnetic signal in order to obtain an encoded electromagnetic signal. While not shown, this embodiment of the invention also preferably includes an encoder. The encoder may for example be embodied be a modulator which applies a modulation on each of the electromagnetic signals 26, the modulation corresponding to the effects data transmitted by the data source 18 and thereby being encoded within the electromagnetic signals. With reference to the embodiment of FIG. 6, the data sent by the data source 18 may be encoded within the electromagnetic signals by the modulator at the time of generating of the electromagnetic by the light emitter. Preferably, in this embodiment the modulator is coupled directly to the light emitter in order to control the emitter such as to directly output the encoded electromagnetic signals.

Alternatively or additionally, as shown in FIG. 45 the modulator may be an external modulator disposed downstream the light emitter and applying the modulation on the electromagnetic signals after they have been generated and outputted by the emitter. For example be embodied by an amplitude modulator, a phase modulator or a more complex modulating system as well known to those skilled in the art. The modulation can be either an analog or a digital modulation. The modulator 42 preferably generates a modulation signal having an amplitude, a frequency and a phase, each of these parameters being possibly controllable to perform the desired modulation. The comm projector 100 can include modulators to modulate the signal of light emitter in one or in a combination of modulation methods. Modulation techniques such amplitude modulation, frequency modulation, phase modulation, phase-shift keying, frequency-shift keyin, on-off keying, spread-spectrum and combinations of these techniques are well known to those skilled in the art. In other embodiments, the encoder may be embodied by other types of devices which act on the electromagnetic signals 26, a filter and/or shutter placed in front of the radiation emitters. Both encoding methods may be used in conjunction. As explained previously, a comm projector 100 can be provided with a single light emitter or combinations of emitters in order to communicate with receiving units 200 using many wavelengths concurrently. Similarly, the signal of a light emitter can be modulated sequentially or concurrently in different ways to communicate different information. Both methods can be used concurrently. In other words, the comm projector 100 can project the electromagnetic signals successively or in parallel, at least for some of the signals.

For example, a comm projector 100 can modulate the signal of an infrared emitter at three different frequencies in order to transmit effects data 18 on three independent channels. Receiving units 200 equipped with amplifiers and/or demodulators tuned to these three frequencies may then change state according to the signal they receive on three independent channels. For example, using red, green and blue LEDs coupled to each of these three associated state color allows the units 200 to display full-color video in real-time. Referring still to FIGS. 5, 6 and 7, the comm projector 100 also includes a projecting module 100 for projecting the electromagnetic signals generated and optionally encoded by the light generating module towards the target locations within the location, as defined by the corresponding coordinate data. Various devices and configurations may be used in the projecting module to spatially direct electromagnetic signals in various directions.

In summary, the present inventions employs multi-communications protocols, including but not limited to wifi, bluetooth, other rf, audio, optical and other em, and projection to direct, spatially distinct, projection of optical data and positional signals, to coordinate a complex visual and audio presentation to any audience, gathering, assembly or manifestation of receivers which may be simultaneously or have previously received data, instructions, settings and commands to present video, audio and other effects.

(Improved Receiver module)

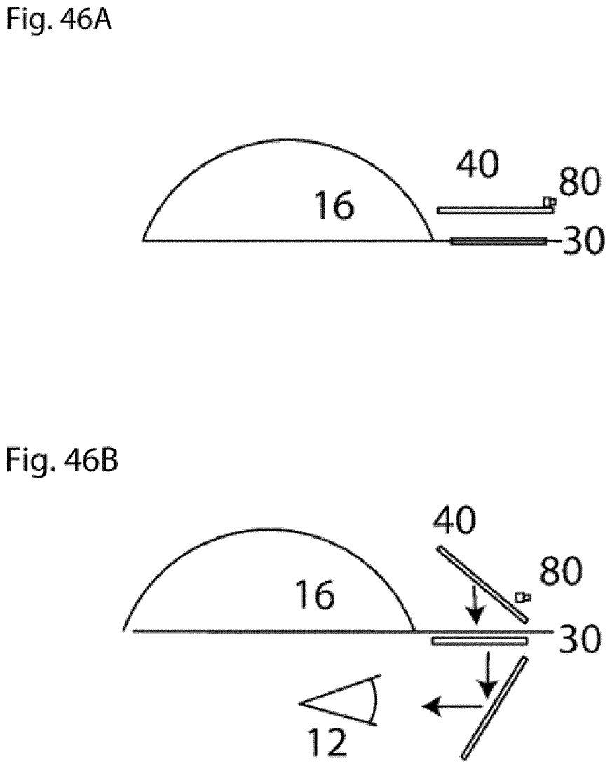

Referring to FIGS. 46A-48B, a preferred embodiment of the audience effects receiver unit 200 is Augmented Reality Receiver Module in the form of an AR Headset as shown in FIGS. 46A and 46B. Said Headsets may be of the “See-Through” AR design enabling safe movement about an environment by a human audience or manifestation, enabled by Smartphone 40 or attached custom receiver modules which may include but are not limited to cameras 80.

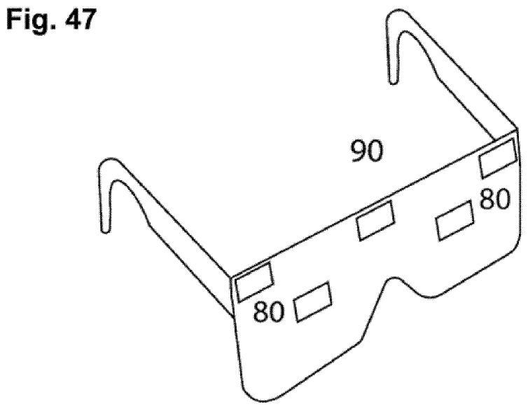

The receiver may have a display module and other effects including LEDs, speakers, and moving, mechanical elements such as is shown in FIGS. 47 and 40B

FIG. 48B shows a perspective view of a game or trade show, ergonomic, front facing display embodiment of the headset device 10 having an exposed, publicly visible region of the display module 640 and a redirect region 641 visible to the user's eye, and wherein the scene visualization means 80 functions as a receiver 200. The scene visualization means 80 may be the integrated ‘selfie’ display-side or light-piped rear camera; or an add-on sensor module such as for IR/UV etc.\

The Augmented Reality Experience may overlay the Environmental Experience, using Camera Registration of real objects, signals or indicators to align the See-Through with the Augmented Experience.

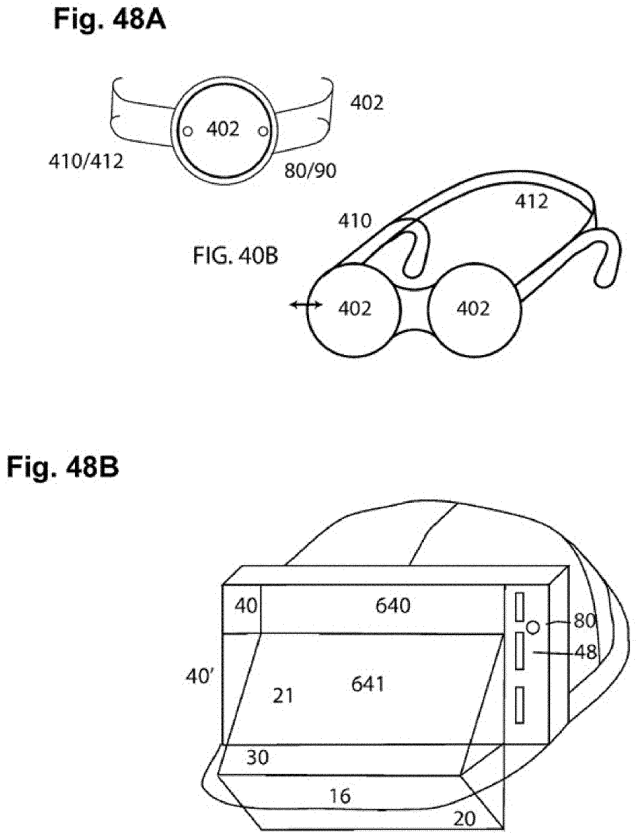

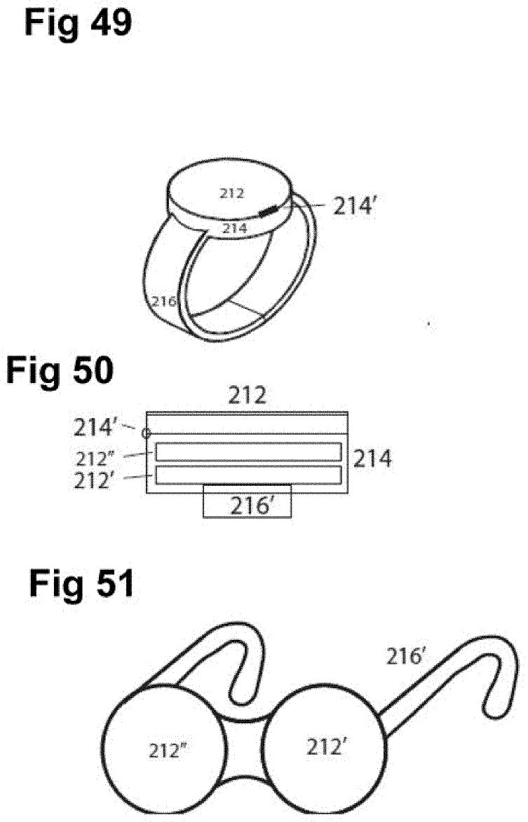

(Application of Collapsible Design to Transformable Wristwatch-EyeGlass) (FIGS. 49-51)

FIGS. 49-51 shows an isometric view of the Wrist embodiment of the present invention having a base cover display 212, a base structure 214 and a base wristband 216. A base structure hinge 214′ is shown which enables the base cover display 212 to open along the line of the arm and orthogonal to the plane of the wristband 216.

FIG. 50 shows an side view along the plane of the wristband-watch temple arms element 216′ having within the base structure 214 a first eyeglass configuration display 212′ and a second eyeglass configuration display 212″. In operation the base structure hinge 214′ enables the base display 212 to open and the eyeglass displays 212′,212″ together the temple arm structures 216′ to be removed and used independently of the base unit 212-214-216. The construction of the eyeglass displays 212′, 212″ is discussed previously but may include multiple, unfoldable elements including a light-emitting matrix, a reflective-transparent mirror lens, one or more transmissive lens elements, one or more reflective mirrors or prisms, or meta-optics.

List of Enumerated Elements

General Specification Concluding Statements

Further, though advantages of the present invention are indicated, it should be appreciated that not every embodiment of the invention will include every described advantage. Some embodiments may not implement any features described as advantageous herein and in some instances. Accordingly, the foregoing description and drawings are by way of example only. Various aspects of the present invention may be used alone, in combination, or in a variety of arrangements not specifically discussed in the embodiments described in the foregoing and is therefore not limited in its application to the details and arrangement of components set forth in the foregoing description or illustrated in the drawings.

For example, aspects described in one embodiment may be 15 combined in any manner with aspects described in other embodiments. The invention may be embodied as a method, of which an example has been described. The acts performed as part of the method may be ordered in any suitable way.

Accordingly, embodiments may be constructed in which acts are performed in an order different than illustrated, which may include different acts than those which are described, and/or which may involve performing some acts simultaneously, even though the acts are shown as being performed sequentially in the embodiments specifically described above. Use of ordinal terms such as “first,” “second,” “third,” etc., in the claims to modify a claim element does not by itself connote any priority, precedence, or order of one claim element over another or the temporal order in which acts of a method are performed, but are used merely as labels to distinguish one claim element having a certain name from another element having a same name (but for use of the ordinal term) to distinguish the claim elements. Also, the phraseology and terminology used herein is for the purpose of description and should not be regarded as limiting. The use of “including,”, “comprising,” or “having,” “containing,” “involving,” and variations thereof herein, is meant to encompass the items listed thereafter and equivalents thereof as well as additional items.

1. A transformable, sensor and display system enabling the rapid transformation from wristwatch form, support and operation to eyeglass form, support and operation; comprising:
a. An articulated eyeglass frame and arms reversibly transformable into a wristwatch form;
b. At least one visual display in the form of a wristwatch viewable at a normal wristwatch distance;
c. At least one visual display in the form of an eyeglass lens viewable at a normal eyeglass lens distance;
d. wherein at least one visual display operationally transforms between wristwatch and eyeglass lens viewable;
e. having at least two sensor elements.

2. A transformable, display system in accordance with claim Ind1, having a transformable display element comprising:
a. an array of sensors
b. said array overlayed by a transformative transmissive to diffusive layer enabling the display to reversibly function as a watch face;
c. and a reversible, external occlusion layer having at least one optical-occlusion controllable section enabling immersive virtual reality.

3. A transformable, display system in accordance with claim 1; further comprising:
a. a foldable, eyeglass bridge component having an elastomeric composition; and
b. a foldable, eyeglass arm components having an elastomeric composition.

4. A transformable display system enabling the rapid transformation from handheld support and operation to head-mounted support and operation, comprising:
a. a head-mountable, compactable headset apparatus conformed to removably affixed and supported by the user's forehead;
b. an easily, removable display device situated above the normal line of sight of the user for the display of visual images and information;
c. a first mirror receiving the image displayed on said display device oriented at an angle approximately 45 degrees to said display device; and
d. a reflective/transmissive eye visor receiving the image from said first mirror and directing the image of said display device to the user's eye, said eye visor having at least one ratio of reflectivity/transmissivity from 100 to 1 percent.

5. A transformable, display system enabling the rapid transformation from handheld support and operation to head-mounted support and operation in accordance with claim 4, further comprising:
a. a cushioning pad removably-situated between the user's forehead and the removable display device;
b. said removable display device having at least one display screen projecting forward from the user's forehead;
c. and being removably-affixed proximal to the forehead of the user.

6. A transformable, display system enabling the rapid transformation from handheld support and operation to head-mounted support and operation in accordance with claim 4, further comprising at least one sensor:
a. a cushioning pad situated between the user's forehead and a cushioning spacer on the head-mounted apparatus;
b. a removable display device situated on the opposing construction of the spacer;
c. the removable display device having at least one display screen projecting away from the user's forehead; and
d. the foam pad and spacer construction being removably-affixed to the forehead of the user.

7. A transformable, display system enabling the rapid transformation from handheld support and operation to head-mounted support and operation in accordance with claim 4, further comprising an integrated transmissive optical element situated above the normal line of the sight of the user and within the optical path between the first mirror and the reflective/transmissive eye visor.

8. A transformable, display system enabling the rapid transformation from handheld support and operation to head-mounted support and operation in accordance with claim 4, further comprising a reversibly insertable, binocular frame having ‘virtual reality’ lenses situated between the user's eyes and the eye visor element.

9. A transformable, display system enabling the rapid transformation from handheld support and operation to head-mounted support and operation in accordance with claim 4, further comprising:
a. a flexible, foldable, forehead-mountable, adjustable head strap which comfortably conforms to the user's forehead; and
b. a foldable, stabilizing device support frame with arms which adjustably connects to the head strap which when properly tensioned with the head strap rigidly affixes the device support frame to the user's forehead, and when released folds compactly within the footprint of the support frame.

10. A transformable, display system enabling the rapid transformation from handheld support and operation to head-mounted support and operation in accordance with claim 4, further comprising:
a. at least one external scene visualization capture component; and
b. at least one optical component adjustably affixed to the capture component enabling the capture of a desired field of view.

11. A transformable, display system enabling the rapid transformation from handheld support and operation to head-mounted support and operation in accordance with claim 4, further comprising:
a. the display device having a display screen projecting forward from the user;
b. the first mirror element and enclosure receiving and reflecting less than the full display screen of said display device creating a reduced AR Optics component;
c. a section of said display screen visible to an external observer whereby an image displayed on said display screen is enabled to contain sections viewed by the user and the external visible accessible section may be viewed by said external observer.

12. A transformable, display system enabling the rapid transformation from handheld support and operation to head-mounted support and operation in accordance with claim 11, further comprised of a section of the display screen visible to an external observer accessible and responsive to at least touch activation.

13. A transformable, display system enabling the rapid transformation from handheld support and operation to head-mounted support and operation in accordance with claim 4, further comprising:
a. at least one physiological sensor enabling the monitoring of the physiological state of the user; and
b. a connected communications component enabling integration of said physiological state.

14. A transformable, display system enabling the integrated rapid transformation from handheld support and operation to head-mounted support and operation, in accordance with claim 4 comprising:
a. an easily transformable base housing a display device generally in the form of a smart phone and enabling the attachment of head-mounting component;
b. an attached, articulated first mirror receiving the image displayed on the screen of said display device oriented at an angle approximately 45 degrees to said display device;
c. an attached, articulated structure enabling the insertion of a transmissive optical component;
d. an attached, articulated reflective/transmissive eye visor receiving the image from said first mirror and directing the image of said display device to the user's eye, said eye visor having at least one ratio of reflectivity/transmissivity from 100 to 1 percent; and
e. wherein the attached, articulated first mirror, structure and eye visor fold flat and upon the backside of the base housing the display device, thereby enclosing the head-mounting component if attached.

15. A transformable, display system, further comprising:
a. a first mirror assembly enabling positioning to conform to the footprint of the transformable base housing;
b. wherein said flat mirror may be positioned for the optimum presentation of the image to the user; and
c. wherein said first assembly may secure the distal sides of the base housing and the optical component structure.

16. A transformable, display system in accordance with claim 15, further comprising:
a. the first mirror element and enclosure receiving and reflecting less than the full display screen of said display device creating a reduced AR Optics component;
b. a section of said display screen visible to an external observer whereby an image displayed on said display screen is enabled to contain sections viewed by the user and the external visible accessible section may be viewed by said external observer; and
c. a section of the display screen visible to an external observer accessible and responsive to at least touch activation.

17. A transformable, display system in accordance with claim 15, further comprising:
a. the first mirror element and enclosure receiving and reflecting less than the full display screen of said display device creating a reduced AR Optics component.

18. A transformable, display system in accordance with claim 15, further comprising:
a. the first mirror element and enclosure receiving and reflecting less than the full display screen of said display device creating a reduced AR Optics component.

19. A transformable, display system in accordance with claim 15, further comprising:
a. the first mirror element and enclosure receiving and reflecting less than the full display screen of said display device creating a reduced AR Optics component.

20. A transformable, display system in accordance with claim 15, further comprising:
a. the first mirror element and enclosure receiving and reflecting less than the full display screen of said display device creating a reduced AR Optics component.
